(19) United States
(12) Patent Application Publication
(10) Pub. No.: US 2009/0048229 A1
(43) Pub. Date: Feb. 19, 2009

(54) Methods for promoting wakefulness

(76) Inventors: Nadia M.J. Rupniak, Cary, NC (US); James F. White, Carlisle, MA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/218,813

(22) Filed: Jul. 18, 2008

Related U.S. Application Data
(60) Provisional application No. 60/961,207, filed on Jul. 18, 2007.

(51) Int. Cl. A61K 31/553; A61K 31/24; A61K 31/55; A61K 31/381; A61P 25/00; A61K 31/4525; A61K 31/407

(52) U.S. Cl. 514/211.13; 514/540; 514/411; 514/217; 514/321; 514/438

(57) ABSTRACT

The present invention relates to methods of promoting wakefulness in an individual by administering a carbamoyl ester or a pharmaceutically acceptable salt thereof.

### RELATED APPLICATION

This application claims the benefit of and priority to U.S. Patent Application No. 60/961,207, filed Jul. 18, 2007, which is incorporated by reference herein.

### BACKGROUND OF THE INVENTION

A variety of substances have been used to promote wakefulness, including for example, caffeine, modafinil, ephedrine, and amphetamines.

Caffeine is one of the most widely used drugs in the world. Caffeine has a wide range of pharmacological effects both desired and undesired, including stimulation of cardiovascular and central nervous systems, diuresis, and relaxation of smooth muscle. Caffeine also produces an increase in systolic and diastolic pressure, especially in combination with psychological stress, as well as a decrease in heart rate. Ingestion of a quantity of caffeine equivalent to one or two cups of coffee causes noticeable physiological effects. Taken before bedtime, caffeine usually delays sleep onset, shortens overall sleep time, and reduces the depth of sleep. After using caffeine, sleepers are more easily aroused, move more during sleep, and report a reduction in the quality of sleep. Larger doses of caffeine, especially when given to non-habitual users cause headaches, jitteriness, and tachycardia. Caffeine elevates neutral activity in many parts of the brain, postpones fatigue, and enhances performance of simple intellectual tasks and of physical work that involves endurance, but caffeine may impair fine motor coordination due to the induction of tremors.

Similarly, modafinil, also known as Provigil®, is a new wakefulness promoting medicine. Modafinil has been approved by the FDA for reducing excess sleepiness in narcolepsy, obstructive sleep apnea/hypopnea syndrome (OSAHS), and shift work sleep disorder (SWSD). The wake-promoting actions of modafinil are similar to sympathomimetic agents like amphetamine and methylphenidate. The precise mechanism(s) through which modafinil promotes wakefulness is unknown. At typical therapeutic concentrations, it is known that modafinil does not bind to certain neurotransmitter receptors that regulate sleep or wakefulness e.g., norepinephrine, serotonin, dopamine, GABA, adenosine, histamine H3, melatonin, or benzodiazepine. Modafinil also does not affect the initiation, maintenance, quality or quantity of nighttime sleep, and it does not affect the ability to sleep voluntarily during the daytime. In addition to its wake-promoting effects, modafinil increases locomotor activity in animals, produces psychoactive and euphoric effects, alterations in mood, perception, thinking, and feelings typical of other CNS stimulants in humans. Modafinil has reinforcing properties, as evidenced by its self-administration in monkeys previously trained to self-administer cocaine. Modafinil was also partially discriminated as psychostimulant-like in animal models. Rash and psychiatric adverse events have been reported in patients treated with modafinil.

Other central nervous system stimulants such as amphetamines, pemoline, methylphenidate, and ephedrine may also be used to promote wakefulness. The potential for abuse of these drugs, however, is well known. Although physical and mental alertness is increased with these drugs, headache, agitation, dysphoria, and fatigue are frequent side-effects, especially as the stimulant effects of the drugs wear off.

A need exists, therefore, for additional wakefulness-promoting medicines that have distinct or superior side-effect profiles, particularly medicines that overcome the shortcomings of existing medicines.

### SUMMARY OF THE INVENTION

The present invention is methods of promoting wakefulness in an individual by administering compounds of the invention. The compounds used in the methods of the invention are carbamoyl esters that have cholinesterase inhibitory activity.

The invention relates to methods of promoting wakefulness in an individual comprising administering to the individual a compound having the formula:

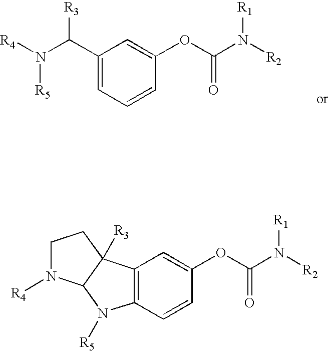

or a pharmaceutically acceptable salt thereof, wherein

R₁ is selected from the group consisting of hydrogen, unsubstituted alkyl, and substituted alkyl;

R₂ is selected from the group consisting of substituted alkyl, unsubstituted aralkyl, substituted aralkyl, unsubstituted heteroaralkyl, substituted heteroaralkyl, unsubstituted heteroaralkyl, substituted heteroaralkyl, unsubstituted aryl, substituted aryl, unsubstituted heteroaryl, substituted heteroaryl, unsubstituted cycloalkyl, substituted cycloalkyl, unsubstituted heterocycloalkyl and substituted heterocycloalkyl; or taken together with the nitrogen atom to which they are attached, R₁ and R₂ form a 5- or 6-membered ring, further wherein the ring is substituted or unsubstituted;

R₃ is selected from the group consisting of hydrogen, unsubstituted alkyl, and substituted alkyl;

R₄ is selected from the group consisting of hydrogen, unsubstituted alkyl, and substituted alkyl; and

R₅ is selected from the group consisting of hydrogen, unsubstituted alkyl, and substituted alkyl.

The invention relates to methods of promoting wakefulness in an individual that suffers from a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy. Wakefulness disorders and conditions include fatigue associated with multiple sclerosis and circadian rhythm disorders such as shift work sleep disorder, sleep apnea, desynchronizing disorder in blind individuals, time zone change syndrome, shift work sleep disorder, irregular sleep pattern, delayed sleep syndrome, and advanced sleep syndrome.

The invention relates to methods of promoting wakefulness in an individual thereby treating the individual for a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy.

The invention relates to a method for enhancing alertness or increasing regularity of sleep rhythms in an individual.

### BRIEF DESCRIPTION OF THE DRAWINGS

FIG. 1 depicts a proposed mechanism of acetylcholinesterase inhibition by the carbamoyl esters of the invention.

FIG. 2 depicts the wake promotion profile of compounds A, B, C, and D in rats.

FIG. 3 depicts the lack of hypersomnolence with compound B in rats.

FIG. 4 depicts the absence of hypothermia of compound B compared with d-amphetamine in rats.

FIG. 5 depicts the lack of locomotor stimulant activity of compound B compared with d-amphetamine in rats.

FIG. 6 depicts the absence of stimulus generalization to compound B in a two-lever drug discrimination procedure using methamphetamine trained rats.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

One aspect of the invention includes a method of promoting wakefulness in an individual comprising administering to the individual a compound having the formula:

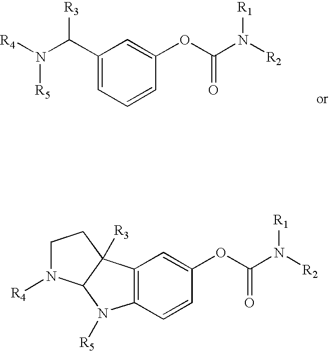

or a salt thereof, wherein

R₁ is selected from the group consisting of hydrogen, unsubstituted alkyl, and substituted alkyl;

R₂ is selected from the group consisting of substituted alkyl, unsubstituted aralkyl, substituted aralkyl, unsubstituted heteroaralkyl, substituted heteroaralkyl, unsubstituted heteroaralkyl, substituted heteroaralkyl, unsubstituted aryl, substituted aryl, unsubstituted heteroaryl, substituted heteroaryl, unsubstituted cycloalkyl, substituted cycloalkyl, unsubstituted heterocycloalkyl and substituted heterocycloalkyl; or taken together with the nitrogen atom to which they are attached, R₁ and R₂ form a 5- or 6-membered ring, further wherein the ring is substituted or unsubstituted;

R₃ is selected from the group consisting of hydrogen, unsubstituted alkyl, and substituted alkyl;

R₄ is selected from the group consisting of hydrogen, unsubstituted alkyl, and substituted alkyl; and

R₅ is selected from the group consisting of hydrogen, unsubstituted alkyl, and substituted alkyl.

In one aspect, the invention includes a method of promoting wakefulness in an individual comprising administering to the individual a compound or salt thereof, having the formula:

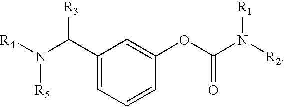

In another aspect, the invention includes a method of promoting wakefulness in an individual comprising administering to the individual a compound or salt thereof, having the formula:

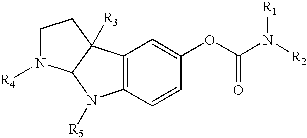

In one aspect, the invention includes a method of promoting wakefulness comprising administering a compound or salt thereof, wherein at least one of R₃, R₄, and R₅ is unsubstituted alkyl. In another aspect, the invention includes administering a compound or pharmaceutically acceptable salt thereof, wherein at least two R₃, R₄, and R₅ are unsubstituted alkyl. In another aspect, the invention includes administering a compound or pharmaceutically acceptable salt thereof, wherein R₃, R₄, and R₅ are each unsubstituted alkyl. In another aspect, the invention includes administering a compound or pharmaceutically acceptable salt thereof, wherein unsubstituted alkyl is methyl.

In one aspect, the invention includes a method of promoting wakefulness comprising administering a compound or salt thereof, wherein the configuration of the stereocenter to which R₃ is attached is in the S-configuration as shown below.

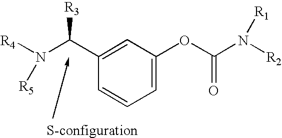

In another aspect, the invention includes a method of promoting wakefulness comprising administering a compound or salt thereof, wherein the configuration of the stereocenter to which R₃ is attached is in the R-configuration as shown below.

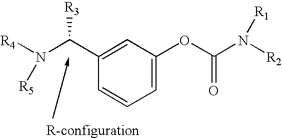

In another aspect, the invention includes a method of promoting wakefulness comprising administering a compound or salt thereof, wherein the configuration of the stereocenter to which R₃ is attached is shown below:

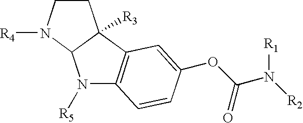

In one aspect, the invention includes a method of promoting wakefulness comprising administering a compound or salt thereof, wherein R₁ is hydrogen. In one aspect, the invention includes administering a compound or salt thereof, wherein R₁ is unsubstituted alkyl. In another aspect, the invention includes administering a compound or salt thereof, wherein R₁ is methyl. In another aspect, the invention includes administering a compound or salt thereof, wherein R₁ is selected from hydrogen and methyl. In one aspect, the invention includes administering a compound or salt thereof, wherein R₁ is substituted alkyl. In another aspect, the invention includes administering a compound or salt thereof, wherein R₁ is alkyl substituted with alkynyl. In another aspect, the invention includes administering a compound or salt thereof, where R₁ is

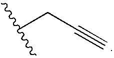

In one aspect, the invention includes a method of promoting wakefulness comprising administering a compound or salt thereof, wherein R₁ and R₂ taken together with the nitrogen atom to which they are attached form a 5- or 6-membered ring. In another aspect, the invention includes a method of promoting wakefulness, comprising administering a compound or salt thereof, wherein R₁ and R₂ taken together with the nitrogen atom to which they are attached form a 6-membered ring. In another aspect, the invention includes administering a compound or salt thereof, wherein the 6-membered ring, formed by R₁ and R₂ and the nitrogen to which they are attached, is substituted with at least one, two, or three substituent(s). In one aspect, the invention includes administering a compound or salt thereof, wherein the 6-membered ring, formed by R₁ and R₂ and the nitrogen to which they are attached, is selected from the group consisting of piperidine and piperazine. In another aspect, the invention includes administering a compound or salt thereof, wherein the 6-membered ring, formed by R₁ and R₂ and the nitrogen to which they are attached, is substituted at the 2-position or 4-position.

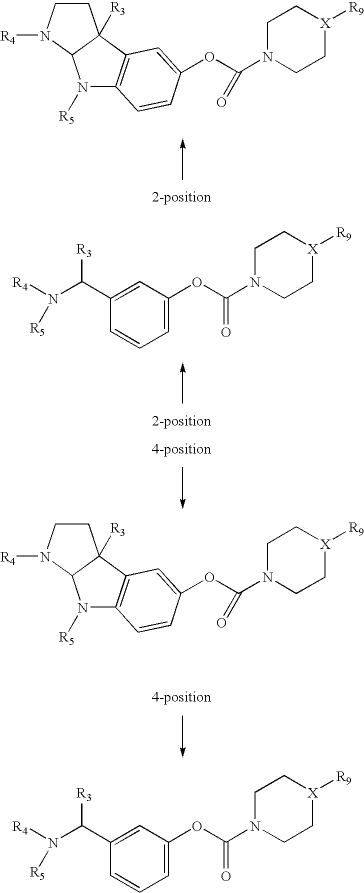

In another aspect, the invention includes administering a compound or salt thereof, wherein the ring, formed by R₁ and R₂ and the nitrogen to which they are attached, is substituted with a moiety containing at least one aromatic ring. In another aspect, the invention includes administering a compound or salt thereof, wherein the ring, formed by R₁ and R₂ and the nitrogen to which they are attached, is substituted with a moiety selected from

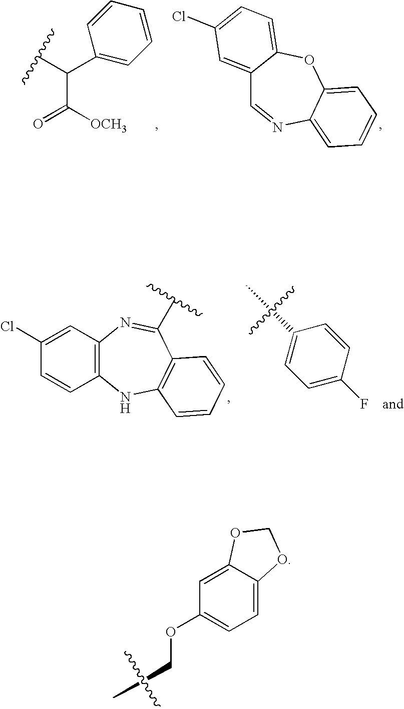

In another aspect, the invention includes administering a compound or salt thereof, wherein the ring, formed by R₁ and R₂ and the nitrogen to which they are attached, is substituted with

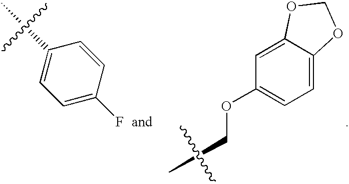

In another aspect, the invention includes administering to an individual a compound or salt thereof, wherein the ring, formed by R₁ and R₂ and the nitrogen to which they are attached, is substituted with a tricyclic ring. In another aspect, the invention includes administering a compound or salt thereof, wherein the ring, formed by R₁ and R₂ and the nitrogen atom to which they are attached, is substituted with

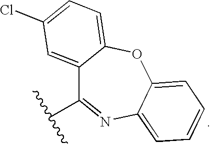

In one aspect, the invention includes administering a compound or salt thereof, wherein R₂ is selected from the group consisting of aralkyl, cycloalkyl, alkyl, and heteroaralkyl, further wherein R₂ is optionally substituted. In another aspect, the invention includes administering a compound or salt thereof, wherein the alkyl moiety of R₂ is aralkyl, alkyl, and heteroaralkyl is two carbon atoms in length. In another aspect, the invention includes administering a compound or salt thereof, wherein the alkyl moiety of R₂ is aralkyl, alkyl, and heteroaralkyl is three carbon atoms in length.

In another aspect, the invention includes administering to an individual a compound or salt thereof, wherein R₂ is substituted with substituted alkyl, unsubstituted alkyl, substituted cycloalkyl, unsubstituted cycloalkyl, substituted aryl, unsubstituted aryl, substituted tricyclic ring, unsubstituted tricyclic ring, substituted alkenyl-tricyclic ring, unsubstituted alkenyl-tricyclic ring, unsubstituted aryloxy, substituted aryloxy, unsubstituted oxime, and substituted oxime.

In one aspect, the invention includes administering a compound or salt thereof, wherein R₂ is substituted aralkyl. In one aspect, the invention includes administering a compound or salt thereof, wherein R₂ is aralkyl substituted with a substituent selected from the group consisting of unsubstituted alkyl and substituted phenoxy. In one aspect, the invention includes administering a compound or salt thereof, wherein R₂ is aralkyl substituted with methyl e.g.,

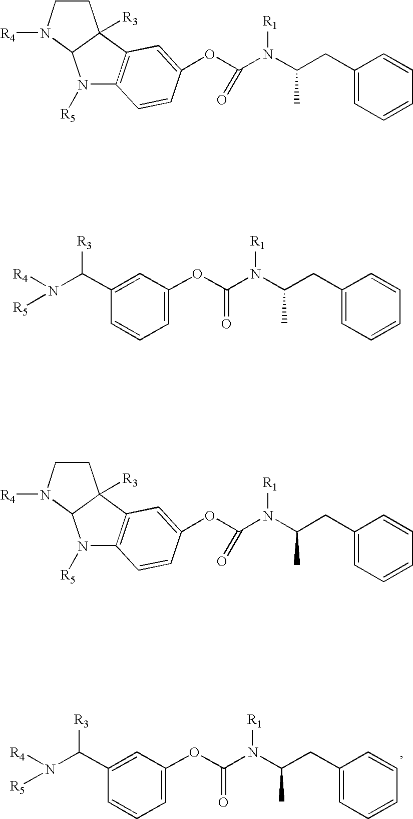

wherein R₁, R₃, R₄ and R₅ are as described herein.

In one aspect, the invention includes administering a compound or salt thereof, wherein R₂ is aralkyl substituted with a substituent selected from the group consisting of

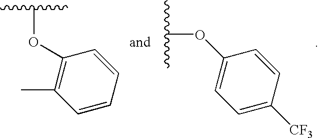

In another aspect, the invention includes administering a compound or salt thereof, wherein R₂ is substituted alkyl. In one aspect, the invention includes administering a compound or salt thereof, wherein R₂ is alkyl substituted with a substituent selected from the group consisting of unsubstituted alkyl, unsubstituted cycloalkyl, unsubstituted tricyclic ring, unsubstituted alkenyl-tricyclic ring, unsubstituted oxime and substituted oxime.

In one aspect, the invention includes administering a compound or salt thereof, wherein R₂ is alkyl substituted with cyclohexyl. In another aspect, the invention includes administering a compound or salt thereof, wherein R₂ is alkyl substituted with

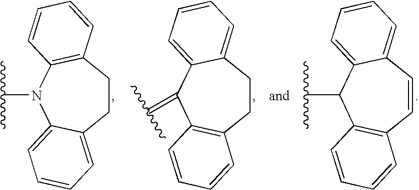

In another aspect, the invention includes a compound or salt thereof, wherein R₂ is alkyl substituted with

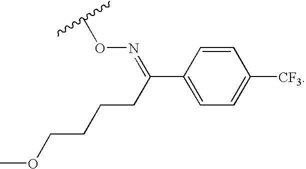

In another aspect, the invention includes administering a compound or salt thereof, wherein R₂ is substituted cycloalkyl. In one aspect, the invention includes administering a compound or salt thereof, wherein R₂ is cyclopropyl. In another aspect, the invention includes administering a compound or salt thereof, wherein R₂ is 1,2,3,4-tetrahydronaphthalene. In one aspect, the invention includes administering a compound or salt thereof, wherein R₂ is cycloalkyl substituted with aryl. In another aspect, the invention includes administering a compound or salt thereof, wherein R₂ is cycloalkyl substituted with a substituent selected from the group consisting of substituted phenyl and unsubstituted phenyl, further wherein phenyl is optionally substituted with at least one halogen. In another aspect, the invention includes administering a compound or salt thereof, wherein R₂ is cycloalkyl substituted with phenyl and phenyl is substituted with at least one chlorine.

In another aspect, the invention includes administering a compound or salt thereof, wherein R₂ is substituted heteroaralkyl. In one aspect, the invention includes administering a compound or salt thereof, wherein the alkyl moiety of heteroaralkyl is substituted with aryloxy. In one aspect, the invention includes administering a compound or salt thereof, wherein aryloxy is

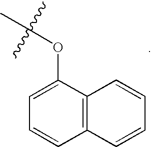

In one aspect, the invention includes a method of promoting wakefulness in an individual comprising administering to the individual a compound of Table 1 or a pharmaceutically acceptable salt thereof. In another aspect, the invention includes a method of promoting wakefulness in an individual comprising administering to the individual a compound selected from Compound 2, 3, 4, 5, 5A, 6, 7, 7A, 8, 9, 9A, 10, 11, 13, 14, 15, 16, 17, 18, 20, 23, and 29.

In one aspect, the invention includes a method of promoting wakefulness in an individual comprising administering to the individual a compound having the formula:

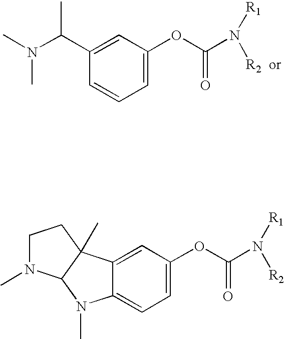

or a salt thereof, wherein

R₁ is selected from the group consisting of hydrogen, unsubstituted alkyl, and substituted alkyl; and

R₂ is selected from the group consisting of substituted alkyl, unsubstituted aralkyl, substituted aralkyl, unsubstituted heteroaralkyl, substituted heteroaralkyl, unsubstituted heteroaralkyl, substituted heteroaralkyl, unsubstituted aryl, substituted aryl, unsubstituted heteroaryl, substituted heteroaryl, unsubstituted cycloalkyl, substituted cycloalkyl, unsubstituted heterocycloalkyl and substituted heterocycloalkyl;

or taken together with the nitrogen atom to which they are attached, R₁ and R₂ form a 5- or 6-membered ring, further wherein the ring is substituted or unsubstituted.

In one aspect, the invention includes a method of promoting wakefulness in an individual comprising administering to the individual a compound having the formula:

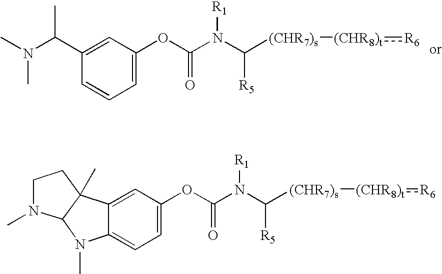

or a salt thereof, wherein
R₁ is selected from the group consisting of hydrogen, unsubstituted alkyl, and substituted alkyl;
R₅ is selected from the group consisting of hydrogen, unsubstituted alkyl, and substituted alkyl; and
R₆ is selected from the group consisting of unsubstituted aryl, substituted aryl, unsubstituted cycloalkyl, substituted cycloalkyl, unsubstituted tricyclic ring, and substituted tricyclic ring;
R₇ is selected from the group consisting of hydrogen, unsubstituted alkyl, and substituted alkyl;
R₈ is selected from the group consisting of hydrogen, unsubstituted alkyl, substituted alkyl, substituted aryloxy, unsubstituted aryloxy; and
s is 0 or 1;
t is 0 or 1, provided that s and t are not both 0; and
-------- is absent or taken together with the bond shown directly above it forms a double bond.

In one aspect, the invention includes a method of promoting wakefulness in an individual comprising administering to the individual a compound having the formula:

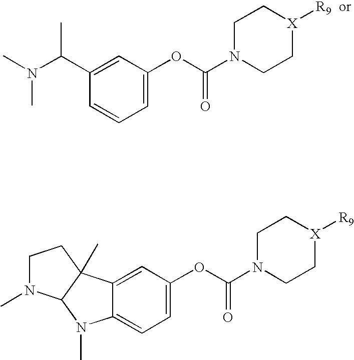

or a pharmaceutically acceptable salt thereof, wherein

### X is N or CH;

R₉ is selected from the group consisting of hydrogen, substituted tricyclic ring, unsubstituted tricyclic ring, substituted aryl, unsubstituted aryl; and
further wherein the piperidine and piperazine ring is optionally substituted.

In another aspect, the invention includes administering a compound, wherein R₉ is

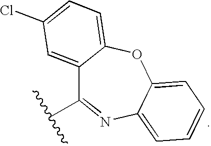

In another aspect, the invention includes administering a compound having the formula:

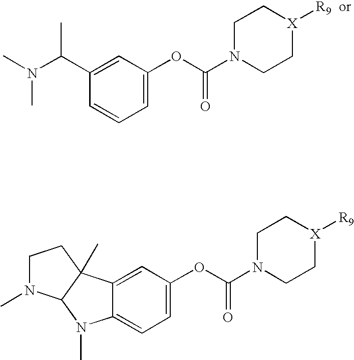

wherein the ring is substituted with

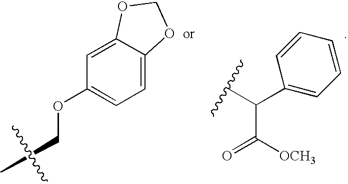

In one aspect, the invention includes a method of promoting wakefulness in an individual, wherein the compound or salt is administered as a pharmaceutical composition including a pharmaceutically acceptable carrier. In another aspect, the invention includes administering a salt, which is a pharmaceutically acceptable salt.

In one aspect, the invention includes a method of promoting wakefulness in an individual, comprising administering to an individual a pharmaceutical composition comprising a compound of Table 1 or a pharmaceutically acceptable salt thereof and a pharmaceutically acceptable carrier.

In another aspect, the invention includes a method of promoting wakefulness in an individual, comprising administering to an individual a pharmaceutical composition comprising a compound selected from 2, 3, 4, 5, 5A, 6, 7, 7A, 8, 9, 9A, 10, 11, 13, 14, 15, 16, 17, 18, 20, 23, and 29 or pharmaceutically acceptable salt thereof and a pharmaceutically acceptable carrier.

One aspect of the invention includes a method of promoting wakefulness in an individual, wherein the individual suffers from a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy.

Another aspect of the invention includes a method of promoting wakefulness, thereby treating the individual for a disorder or condition selected from a wakefulness disorder, hypersomnia, sleep apnea, sleep disorder of central origin, fatigue, excessive daytime sleepiness associated with narcolepsy, fatigue and excessive sleepiness associated with a major depressive disorder or with antidepressant therapy.

In one aspect, the invention includes a method for the treatment of a wakefulness disorder by administering to an individual a carbamoyl ester as a wake promoting agent. In one aspect, the invention includes a method for the treatment of sleep apnea by administering to an individual a carbamoyl ester as a wake promoting agent. In one aspect, the invention includes a method for the treatment of a sleep disorder of central origin by administering to an individual a carbamoyl ester as a wake promoting agent. In one aspect, the invention includes a method for the treatment of fatigue by administering to an individual a carbamoyl ester as a wake promoting agent. In one aspect, the invention includes a method for the treatment of excessive daytime sleepiness associated with narcolepsy by administering to an individual a carbamoyl ester as a wake promoting agent. In one aspect, the invention includes a method for the treatment fatigue and excessive sleepiness associated with a major depressive disorder by administering to an individual a carbamoyl ester as a wake promoting agent. In one aspect, the invention includes a method for the treatment fatigue and excessive sleepiness associated with antidepressant therapy.

Fatigue and excessive sleepiness are among the symptoms of a major depressive disorder, and can be adverse experiences associated with antidepressant therapy and are often residual symptoms inadequately treated with SSRI antidepressant therapy. Antidepressant therapy includes but is not limited to therapy with the following antidepressants: tricyclic antidepressants, selective serotonin reuptake inhibitors (SSRIs), serotonin and noradrenaline reuptake inhibitors, monoamine oxidase inhibitors and monoamine oxidase type A. In another aspect, antidepressant is selected from citalipram, fluoxetine, fluoxetine hydrochloride, paroxetine, paroxetine hydrochloride, and clominpramine hydrochloride.

In one aspect, the invention relates to hypersomnia, a condition that is characterized by reoccurring episodes of excessive daytime sleepiness (EDS) or prolonged nighttime sleep. Different from feeling tired due to lack of or interrupted sleep at night, persons with hypersomnia are compelled to nap repeatedly during the day, often at inappropriate times such as at work, during a meal, or in conversation. These daytime naps usually provide no relief from symptoms. Patients often have difficulty waking from a long sleep, and may feel disoriented. Other symptoms may include anxiety, increased irritation, decreased energy, restlessness, slow thinking, slow speech, loss of appetite, hallucinations, and memory difficulty. Some patients lose the ability to function in family, social, occupational, or other settings. In one aspect, the invention includes a method for the treatment of hypersomnia, which comprises administering to an individual a carbamoyl ester as a wake promoting agent. In another aspect, the invention includes a method for the treatment of hypersomnia, which comprises administering to an individual a carbamoyl ester as an arousing agent.

In another aspect, the invention includes a method of promoting wakefulness, wherein the wakefulness disorder or condition is selected from circadian rhythm disorder and fatigue associated with multiple sclerosis.

In one aspect, the invention includes a method of promoting wakefulness, wherein the circadian rhythm disorder is selected from shift work sleep disorder, sleep apnea, desynchronizing disorder in blind individuals, time zone change syndrome, shift work sleep disorder, irregular sleep pattern, delayed sleep syndrome, and advanced sleep syndrome. In another aspect, the invention includes a method of promoting wakefulness, wherein the circadian rhythm disorder is selected from shift work sleep disorder, sleep apnea, and desynchronizing disorder in blind individuals.

In one aspect, the invention relates to sleep apnea. Sleep apnea is a sleep disorder characterized by pauses in breathing during sleep. Each episode, called an apnea, lasts long enough so that one or more breaths are missed, and such episodes occur repeatedly throughout sleep. The standard definition of any apneic event includes a minimum 10 second interval between breaths, with either a neurological arousal (a 3-second or greater shift in EEG frequency, measured at C3, C4, O1, or O2), a blood oxygen desaturation of 3-4% or greater, or both arousal and desaturation. Sleep apnea is diagnosed with an overnight sleep test called a polysomnogram.

Clinically significant levels of sleep apnea are defined as five or more episodes per hour of any type of apnea (from the polysomnogram). There are three distinct forms of sleep apnea: central, obstructive, and complex (i.e., a combination of central and obstructive) constituting 0.4%, 84% and 15% of cases respectively. Breathing is interrupted by the lack of respiratory effort in central sleep apnea; in obstructive sleep apnea, breathing is interrupted by a physical block to airflow despite respiratory effort. In complex (or “mixed”) sleep apnea, there is a transition from central to obstructive features during the events themselves.

In one aspect, the invention includes a method for the treatment of sleep disorders of central origin by administering to an individual a carbamoyl ester. In another aspect, the invention includes a method for the treatment of sleep disorders of central origin by administering to an individual a carbamoyl ester, wherein the number of apneas occurring during sleep apnea syndromes is reduced. In one aspect, treatment of sleep disorders of central origin by administering a carbamoyl ester contributes to improving diurnal somnolence and the quality of nocturnal sleep.

In one aspect, the invention includes a method of promoting wakefulness in an individual, wherein individual is being treated for sleep apnea with CPAP. “CPAP” or “continuous positive airway pressure” is a mechanical device for the treatment for sleep apnea and other sleep-related breathing disorders (including snoring). Treatment with a CPAP device is typically administered via the nose or mouth of the patient.

Under CPAP treatment, a subject wears a tight-fitting plastic mask over the nose when sleeping. The mask is attached to a compressor, which forces air into the nose creating a positive pressure within the subject's airways. The principle of the method is that pressurizing the airways provides a mechanical “splinting” action, which prevents or lessens airway collapse and therefore, obstructive sleep apnea. Although an effective therapeutic response is observed in most subjects who undergo CPAP treatment, many subjects cannot tolerate the apparatus or pressure and refuse treatment. Moreover, recent covert monitoring studies demonstrated that long-term compliance with CPAP treatment is very poor. It is known that subjects remove their mask while sleeping.

In another aspect, the invention relates to fatigue associated with multiple sclerosis (MS). Multiple sclerosis is one of the most common disabling neurologic diseases of young adults in the United States, where an estimated 400,000 persons have the disease. Although MS can cause a variety of disabling neurological impairments such as blindness, paralysis, incoordination, and bowel or bladder dysfunction, a less apparent symptom that can also be severely disabling is fatigue. As used herein “fatigue” includes loss of power, or capacity to respond to stimulation. Effect treatment of such fatigue includes alleviating tiredness, or sleepiness associated with multiple sclerosis and also promoting wakefulness in multiple sclerosis individuals. The mechanism of MS fatigue is poorly understood. It has been attributed to nerve conduction abnormalities within the central nervous system and increased energy demands caused by neurologic disability. Several characteristics of MS fatigue are interference with physical functioning and activities of daily living, aggravation by heat, and worsening at the end of the day. In aspect, the invention includes a method of treatment for fatigue associate with multiple sclerosis, comprising administering to an individual a carbamoyl ester in an amount effective to improve or prevent symptoms of multiple sclerosis fatigue in the individual. In another aspect, the invention includes alleviating tiredness, or sleepiness associated with multiple sclerosis and also promoting wakefulness in multiple sclerosis individuals.

One aspect of the invention includes a method for enhancing alertness or increasing regularity of sleep rhythms in an individual comprising administering to the individual a compound having the formula:

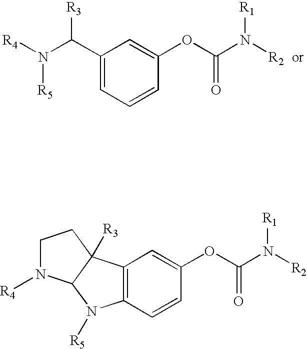

or a pharmaceutically acceptable salt thereof,

R₁ is selected from the group consisting of hydrogen, unsubstituted alkyl, and substituted alkyl;

R₂ is selected from the group consisting of substituted alkyl, unsubstituted aralkyl, substituted aralkyl, unsubstituted heteroaralkyl, substituted heteroaralkyl, unsubstituted heteroaralkyl, substituted heteroaralkyl, unsubstituted aryl, substituted aryl, unsubstituted heteroaryl, substituted heteroaryl, unsubstituted cycloalkyl, substituted cycloalkyl, unsubstituted heterocycloalkyl and substituted heterocycloalkyl;

or taken together with the nitrogen atom to which they are attached, R₁ and R₂ form a 5- or 6-membered ring, further wherein the ring is substituted or unsubstituted;

R₃ is selected from the group consisting of hydrogen, unsubstituted alkyl, and substituted alkyl;

R₄ is selected from the group consisting of hydrogen, unsubstituted alkyl, and substituted alkyl; and

R₅ is selected from the group consisting of hydrogen, unsubstituted alkyl, and substituted alkyl.

In one aspect, the invention includes a method of promoting wakefulness, wherein the compound or salt thereof administered has a reduced abuse potential. In one aspect of the invention, no psychostimulant-like effects are observed in the individual following administration of the compound or salt. A psychostimulant is a drug that causes a sense of well-being, decreases fatigue and depression, and increases the desire to eat. Psychostimulant drugs can also cause mood changes and trouble with sleeping. In another aspect of the invention, the compound or pharmaceutically acceptable salt thereof administered has a dose-limiting side effect. In one aspect, of the invention, the side effect is nausea.

In another aspect of the invention, administration of the compound or salt does not cause rebound hypersomnolence in the individual. The term “hypersomnolence” refers to an excessive need for sleep, especially during the day. “Idiopathic hypersomnolence” means a need for excessive daytime sleep without a known cause. In another aspect of the invention, administration of the compound or a pharmaceutically acceptable salt thereof does not cause hyperthermia in the individual The term “hyperthermia” refers to an increase in body temperature. In another aspect of the invention, administration of the compound or a salt thereof may cause hypothermia in the individual. The term “hypothermia” refers to a fall in body temperature. In one aspect, the fall in temperature in the individual is ≧0.5° C. In another aspect of the invention, administration of the compound or pharmaceutically acceptable salt does not cause locomotor hyperactivity i.e., administration of the compound or a pharmaceutically acceptable salt does not cause the subject to increase movement from place to place.

In one aspect, the invention includes administering an effective amount of the compound or salt. In one aspect, the salt is a pharmaceutically acceptable salt. In another aspect, the invention includes a method of promoting wakefulness, wherein the compound or pharmaceutically acceptable salt thereof is administered to an individual in need of treatment thereof.

In one aspect, the invention includes, wherein the compound or pharmaceutically acceptable salt thereof is administered enterally, parenterally, orally or intramuscularly. In one aspect, the invention includes a method of promoting wakefulness in an individual by administering a compound or salt thereof, wherein the minimum effective dose (MED) of the compound or salt is ≦8 mg/kg p.o.

One aspect of the invention includes a kit for carrying out the method of promoting wakefulness in an individual as described herein.

In another aspect, the invention includes the use of compound or salt thereof, having a formula selected from

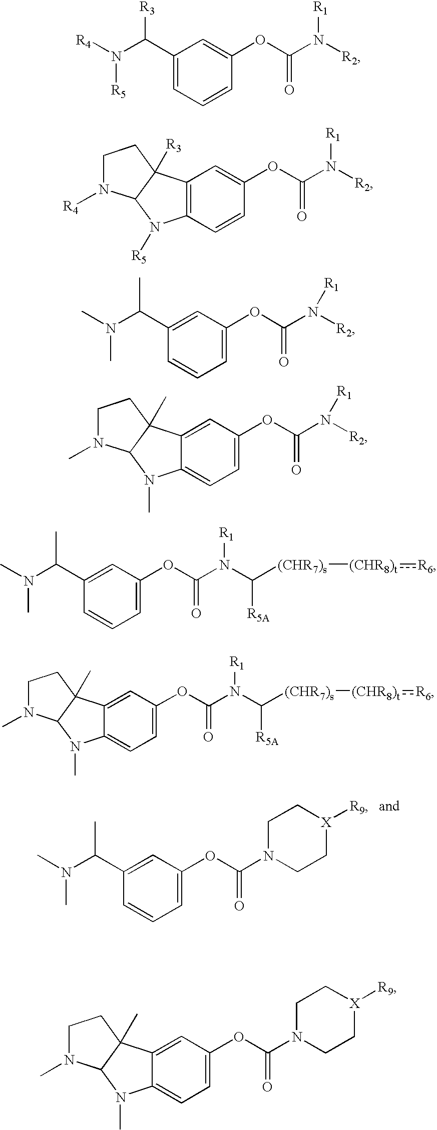

wherein R₁, R₂, R₃, R₄, R₅, R_{5A}, R₆, R₇, R₈, s, t, --------, X and R₉ are as described herein in the manufacture of a medicament for promoting wakefulness in an individual.

In one aspect, the invention includes the use of a compound of Table 1 or salt thereof, in the manufacture of a medicament for promoting wakefulness in an individual.

Another aspect of the invention includes the manufacture of a medicament for promoting wakefulness in an individual that suffers from a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy.

Another aspect of the invention includes the manufacture of a medicament for promoting wakefulness in an individual and thereby treating the individual a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy.

In one aspect, the invention includes the use of a compound selected from Compound 2, 3, 4, 5, 5A, 6, 7, 7A, 8, 9, 9A, 10, 11, 13, 14, 15, 16, 17, 18, 20, 23, and 29 or salt thereof, in the manufacture of a medicament for promoting wakefulness in an individual.

In another aspect, the invention includes a method of promoting wakefulness by administering a compound that inhibits acetylcholinesterase. In one aspect, the invention includes wherein the compound selectively inhibits acetylcholinesterase over butyrylcholinesterase. In one aspect, the compound is at least 3-fold, 4-fold, 5-fold, 8-fold, or 10-fold more selective for acetylcholinesterase over butyrylcholinesterase.

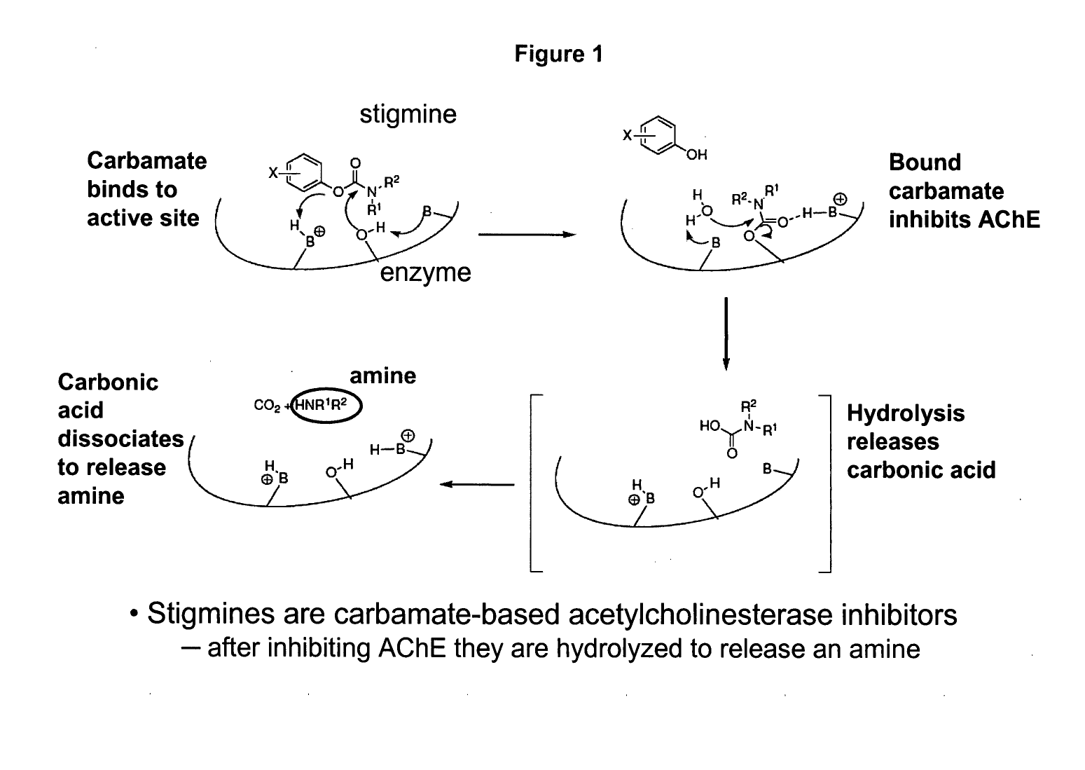

In particular, it is thought that the carbamoyl ester inhibits a cholinesterase by competing with a compound (e.g., acetylcholine (ACh)) that binds to the cholinesterase. As shown in FIG. 1, the carbamoyl ester binds to the cholinesterase to form a carbamoylated enzyme. The cholinesterase is inhibited when it is prevented from inactivating a compound, such as the neurotransmitter ACh, to any degree that cholinesterase would act on the neurotransmitter in the absence of the carbamoyl ester. Hydrolysis of the carbamoylated enzyme is much slower than that of, for example, an acetylated enzyme, which is formed by hydrolysis of its endogenous substrate acetylcholine. Inhibition of the cholinesterase by a carbamoyl ester molecule ceases when the carbamoylated enzyme is hydrolyzed. Upon hydrolysis of the carbamoylated enzyme, a released compound, such as an amine, becomes at least a component of a pharmacologically active agent.

Hydrolysis of the carbamoyl ester comprising an amine group, to become at least a component of a pharmacologically active agent, can be hydrolysis by an enzyme (e.g., a cholinesterase) or hydrolysis by other than an enzyme, such as by an acid (e.g., gastric acid). In one embodiment, the carbamoyl ester that inhibits a cholinesterase, comprises an amine group that, upon hydrolysis by reaction with the cholinesterase, becomes at least a component of a pharmacologically active agent.

The phrase “upon hydrolysis by reaction with an enzyme,” as used herein, refers to the two-step process of reaction of the carbamoyl ester with an enzyme to form a carbamoylated enzyme, and decomposition of the carbamoylated enzyme by reaction with H₂O.

Likewise, the phrase “upon hydrolysis by reaction with the cholinesterase,” as used herein, refers to the two-step process of reaction of the carbamoyl ester with the enzyme cholinesterase, to form a carbamoylated enzyme, and decomposition of the carbamoylated enzyme by reaction with H₂O.

The cholinesterase inhibited by the carbamoyl ester of the invention can be, for example, at least one member selected from the group consisting of an acetylcholinesterase (AChE) or a butyrylcholinesterase (BuChE). The carbamoyl ester can inhibit ACHE alone, BuChE alone, or can inhibit both AChE and BuChE to similar or different degrees.

ACHE is located on excitable membranes and inactivates ACh. The excitable membrane can be a presynaptic neuron or a postsynaptic neuron. ACHE is also referred to as specific cholinesterase. BuChE is located on excitable membranes and non-neuronal tissue such as blood cells (Darvesh, S. et al., Nature Reviews 4: 131-138 (2003), the teachings of which are hereby incorporated by reference in its entirety). BuChE is also referred to as pseudocholinesterase or nonspecific cholinesterase. ACHE and BuChE are regulators of cholinergic neurotransmission in the central nervous system (brain and spinal cord), peripheral nervous system and autonomic nervous system (parasympathetic nervous system and sympathetic nervous system).

Upon hydrolysis of the carbamate bond of the carbamoylated enzyme, a released compound, such as a compound that includes an amine, becomes at least a component of a pharmacologically active agent. The term “becomes at least a component of a pharmacologically active agent,” as used herein, refers to the release of a compound, such as an amine-containing compound, as a consequence of hydrolysis of the carbamoylated enzyme. The compound released by hydrolysis of the carbamoylated enzyme is at least a portion of a pharmacologically active agent. In one embodiment, the compound released by the hydrolysis of the carbamoylated enzyme is a prodrug. The term “prodrug,” as used herein, refers to a compound, such as a carbamoyl ester of the invention, that is administered, but is not the actual drug desired in the treatment regimen and is transformed by metabolic processes to the actual drug desired in the treatment. The prodrug then can be modified to release a pharmacologically active agent. In another embodiment, the compound released by hydrolysis of the carbamoylated enzyme can, itself, be the pharmacologically active agent. Thus, a carbamoyl ester of the invention has a dual role as an inhibitor of a cholinesterase and as a delivery vehicle for a pharmacologically active agent.

The term “pharmacologically active agent,” as used herein, refers to a compound that influences biological processes by altering the activity, localization and/or expression of molecules (e.g., neurotransmitters, peptides, proteins) which are directly or indirectly involved in the biological processes.

The term “locomotor activity” refers to the movement from place to place. In psychophamacology, locomotor activity of lab animals is often monitored to assess the behavioural effects of these drugs. Locomotor activity is useful and often used in primary evaluations of drugs.

The term “compounds of the invention” refers to the carbamoyl esters used in the methods of the invention and described herein. Carbamoyl esters used in the methods of the invention are compounds that contain a carbamate functional group e.g., —OC(O)NH—; an N-alkyl carbamate —OC(O)N(alkyl), wherein alkyl is optionally substituted; —OC(O)NR₁R₂, wherein R₁ and R₂ form a ring, wherein the ring is optionally substituted.

The term “substituted,” as used herein, means that any one or more hydrogens on the designated atom is replaced with a selection from the indicated group, provided that the designated atom's normal valency is not exceeded, and that the substitution results in a stable compound.

A chemical structure showing a dotted line representation for a chemical bond indicates that the bond is optionally present. For example, a dotted line drawn next to a solid single bond indicates that the bond can be either a single bond or a double bond.

The term “alkyl,” used alone or as part of a larger moiety, includes both straight, branched, or cyclic saturated hydrocarbon chains containing one to twelve carbon atoms. The term “lower alkyl” means C₁₋₆ alkyl and is intended to include C₁, C₂, C₃, C₄, C₅, and C₆ alkyl groups.

As used herein, “cycloalkyl” is intended to include saturated ring groups, such as cyclopropyl, cyclobutyl, or cyclopentyl. C₃₋₈ cycloalkyl is intended to include C₃, C₄, C₅, C₆, C₇, and C₈ cycloalkyl groups.

As used herein, “halo” or “halogen” refers to fluoro, chloro, bromo, and iodo substituents.

A “heteroalkyl”, as used herein, is an alkyl group in which one or more carbon atoms is replaced by a heteroatom.

The term “aryl,” used alone or as part of a larger moiety as in “aralkyl” or “aralkoxy,” are carbocyclic aromatic ring systems (e.g. phenyl), fused polycyclic aromatic ring systems (e.g., naphthyl and anthracenyl) and aromatic ring systems fused to carbocyclic non-aromatic ring systems (e.g., 1,2,3,4-tetrahydronaphthyl and indanyl) having five to about fourteen carbon atoms.

The term “heteroaryl,” used alone or as part of a larger moiety as in “heteroaralkyl” or “heteroarylalkoxy,” refers to aromatic ring system having five to fourteen members and having at least one heteroatom. Preferably a heteroaryl has from one to about four heteroatoms. Preferred heteroalkyls are those wherein the heteroatom is selected from the groups consisting of oxygen, sulfur, nitrogen, phosphorase and halides. Examples of heteroaryl rings include pyrazolyl, furanyl, imidazolyl, isoxazolyl, oxadiazolyl, oxazolyl, pyrrolyl, pyridyl, pyrimidinyl, purinyl, pyridazinyl, pyrazinyl, thiazolyl, thiadiazolyl, isothiazolyl, triazolyl, thienyl, 4,6-dihydro-thieno[3,4-c]pyrazolyl, 5,5-dioxide-4,6-dihydrothieno[3,4-c]pyrazolyl, thianaphthenyl, 1,4,5,6-tetrahydrocyclopentapyrazolyl, carbazolyl, benzimidazolyl, benzothienyl, benzofuranyl, indolyl, azaindolyl, indazolyl, quinolinyl, benzotriazolyl, benzothiazolyl, benzothiadiazolyl, benzooxazolyl, benzimidazolyl, isoquinolinyl, isoindolyl, acridinyl, and benzoisazolyl. Preferred heteroaryl groups are pyrazolyl, furanyl, pyridyl, quinolinyl, indolyl and imidazolyl.

An “aralkyl” group, as used herein, is an aryl substituent that is linked to a compound by a straight chain or branched alkyl group having from one to twelve carbon atoms. In one aspect, the aryl substituent is linked to a compound by a straight chain or branched alkyl group having 1-6 carbon atoms i.e., a lower alkyl group. The alkyl moiety of the aralkyl group is optionally substituted.

A “heterocycloalkyl” or “(heterocycle)alkyl” group, as used herein, is a heterocycle substituent that is linked to a compound by a straight chain or branched alkyl group having from one to twelve carbon atoms. In one aspect, the heterocycle substituent is linked to a compound by a straight chain or branched alkyl group having 1-6 carbon atoms i.e., a lower alkyl group. The alkyl moiety of the heterocycloalkyl or (heterocycle)alkyl group is optionally substituted.

A “heteroaralkyl” group, as used herein, is a heteroaryl substituent that is linked to a compound by a straight chain or branched alkyl group having from one to twelve carbon atoms. In one aspect, the heteroaryl substituent is linked to a compound by a straight chain or branched alkyl group having 1-6 carbon atoms i.e., a lower alkyl group. The alkyl moiety of the heteroaralkyl group is optionally substituted.

An aryl (including aralkyl, aralkoxy and the like) or heteroaryl (including heteroaralkyl and heteroaralkoxy and the like) may contain one or more substituents. Examples of suitable substituents include aliphatic groups, aryl groups, haloalkoxy groups, heteroaryl groups, halo and hydroxy.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent carbamoyl ester is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., USA, p. 1445 (1990).

Methods to prepare the carbamoyl esters of the invention, such as aromatic carbamoyl esters, are within the knowledge of one skilled in the art (see, for example, U.S. Pat. Nos. 5,665,880; 5,677,457; and WO 97/14694, the teachings of which are hereby incorporated by reference in their entirety).

In one embodiment, synthesis of aromatic carbamoyl esters can be accomplished by activation of an amine group of a compound to form an activated amine. The activated amine can be isolated and reacted with a phenol group of another compound to form the carbamoyl ester. For example, a primary amine can be converted into an isocyanate. Alternatively, amines can be converted into carbamoyl chlorides. Amines can also be activated and used in situ for the formation of the carbamoyl ester, such as by reacting an amine with activating agents that contain carbonyl chlorides (e.g. phosgene, triphosgene), by reacting the amine with activating agents that contain nitrophenyloxycarbonyl groups (e.g. bis-4-nitrophenylcarbonate, 4-nitrophenylchloroformate), or by reacting the amine with carbonyldiimidazole. The individual steps of amine activation and formation of the carbamoyl ester can be catalyzed by a variety of agents, such as acids, bases, and nucleophiles, separately or in combination.

In another embodiment, synthesis of the carbamoyl esters can be accomplished by activation of a phenol group of a compound to form an activated phenol. The activated phenol is reacted with an amine group of another compound. Activation of the phenol can be performed in a variety of ways, such as by reacting the phenol with activating agents that contain carbonyl chlorides (e.g., phosgene, triphosgene), by reacting the phenol with activating agents that contain nitrophenyloxycarbonyl groups (e.g., bis-4-nitrophenylcarbonate, 4-nitrophenylchloroformate), or by reacting the phenol with carbonyldiimidazole. The individual steps of phenol activation and formation of the carbamoyl ester can be catalyzed by a variety of agents, such as acids, bases, and nucleophiles, separately or in combination.

The carbamoyl esters can be analyzed by well-known analytical methods, including NMR.

Carbamoyl esters can be synthesized, for example, by reaction of the phenolic hydroxyl group in eseroline with carbonyldiimidazole (CDI) in ethylacetate followed by addition of acetic acid and the amine resulted in formation of the aromatic carbamoyl ester (Gao et al., J. Heterocyclic Chem 37:331-333 (2000), the teachings of which are hereby incorporated by reference in their entirety).

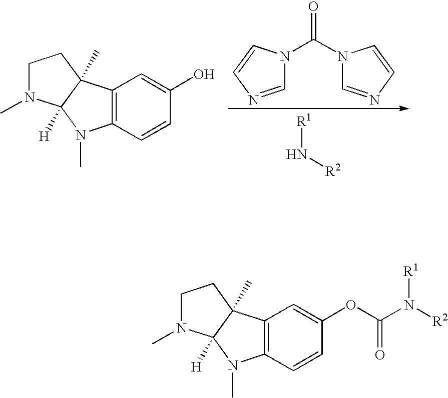

Formation of aromatic carbamoyl esters from eseroline has been described using carbamoyl chlorides (Marta, et al., Bichimica et Biophysica Acta 1120:262-266 (1992); Marta, et al., Biomed Biochem Acta 47:285-288 (1998); Marta, et al., Life Sci. 43:1921-1928 (1988), the teachings of which are hereby incorporated by reference in their entirety).

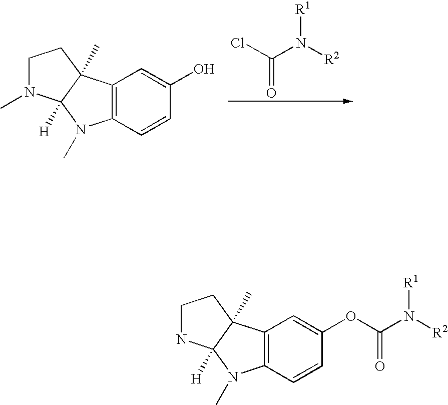

Reaction of a phenolic hydroxyl group with carbamoyl chlorides has also been described for the synthesis of aromatic carbamoyl esters (Toda, et al., Bioorg Med Chem 11:1935-1955 (2003), Kogen, et al., Org Lett 4:3359-3362 (2002), Mustazza, et al., Eur J. Med Chem 37:91-109 (2002) and Sterling, et al., J Med Chem 45:5260-5279 (2002), the teachings of all of which are hereby incorporated by referenced in their entirety).

The term “wake promotion” or “promoting wakefulness” as used herein, refers to a marked increase in the duration of wakefulness of an individual. In one aspect, there is no rebound hypersonmolence in an individual to whom a compound of the invention is administered. In another aspect, hypothermia exhibited by an individual is used as a marker of CNS penetration by a compound administered to an individual for wake promotion. In one aspect, there is a reduction in drowsiness i.e., there is an increased state of mental alertness, or the prevention of further progression into a deeper state of drowsiness that prefaced administration of the carbamoyl ester. The term “drowsiness” is art-recognized, including decreased states of mental alertness.

The term “amphetamine,” such as is used when referring to “1-amphetamine” and “d-amphetamine,” means a compound represented by Formula XXII, including prodrugs and other structural and functional derivatives thereof wherein the primary amine group is available for substitution. In a preferred embodiment, the amphetamine is the compound represented by Formula XXII:

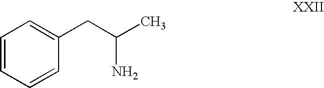

The dextro enantiomer of amphetamine is referred to as the d, (+), D or S isomer and is represented by the following structural formula:

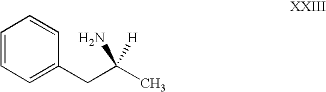

The levo enantiomer of amphetamine can be referred to as the 1, (−), L or R and is represented by the following structural formula:

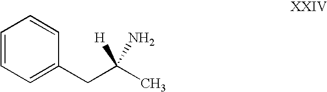

Racemic mixtures of d-amphetamine and 1-amphetamine are referred to as d1, (+,−), (Å), or DL or (R)(S).

An (R)-(−)-amphetamine employed in the methods of the invention is represented by the structural formula:

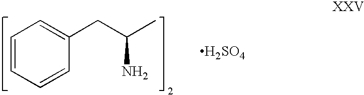

Formula XXV is also referred to as levo-amphetamine sulfate or 1-amphetamine sulfate. Formula XXV has the molecular formula C₁₈H₂₈N₂O₄S and a molecular weight of 368.50. The IUPAC chemical name of Formula XXV is (−)-1-methyl-2-phenylethylamine sulfate (2:1) and the CAS chemical name (−)-_-methylphenethylamine sulfate (2:1).

An (S)-(−)-amphetamine employed in the methods of the invention is represented by the structural formula:

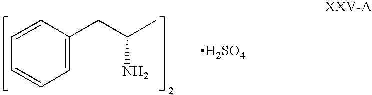

The term “methamphetamine,” such as is used when referring to “1-methamphetamine” and “d-methamphetamine,” means a compound represented by Formula XXVI

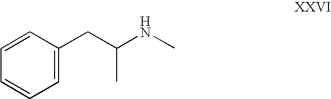

The (R)-(−)-methamphetamine can be represented by the structural formula:

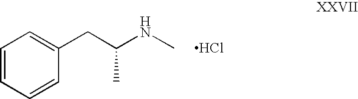

Formula XXVII is also referred to levo-methamphetamine HCl, 1-methamphetamine HCl or levomethamphetamine HCl. Formula XXVII has the molecular formula C₁₀H₁₆NCl.

The (S)-(−)-methamphetamine can be represented by the structural formula:

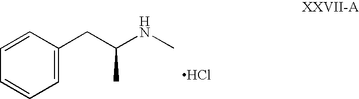

In still another embodiment, the (R)-(−)-methamphetamine can be represented by the structural formula:

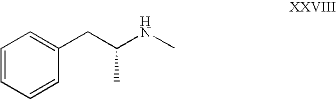

Formula XXVIII is also referred to levo-methamphetamine, levo-desoxyephedrine, 1-desoxyephedrine or levmetamfetamine. Formula XXVIII has the molecular formula C₁₀H₁₅N and a molecular weight of 149.24.

In still another embodiment, the (S)-(−)-methamphetamine can be represented by the structural formula:

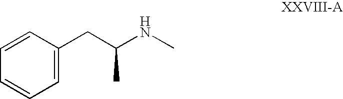

An “agent,” as used herein, refers to a compound that can produce a physical, chemical or biological effect that can be stimulatory (e.g., an activating agent) or inhibitory (e.g., a blocking agent). Agents that are stimulatory can be agonists. Agents that are inhibitory can be antagonists or inverse agonists. Inverse agonists are compounds or molecules that down-regulate receptor activated activity thereby acting in a manner that is the opposite of an agonist to the receptor. Agents can be partial agonists. Thus, exposure or administration of an inverse agonist or partial inverse agonist can result in a diminished response compared to exposure or administration of an agonist.

A “modulator,” as used herein, refers to a compound that regulates, adjusts or adapts a biological pathway or receptor-mediated signal transduction pathway. The modulators can stimulate or inhibit a biological pathway or receptor-mediated signal transduction pathway.

The pharmacologically active agent released by the carbomyl ester is at least one member selected from the group consisting of a symathomimetic agent, an adrenergic agent, a noradrenergic agent, a dopaminergic agent, a serotonergic agent, a mono-amine oxidase inhibitor, and a COMT inhibitor.

The carbamoyl ester of the invention can inhibit cholinesterase activity, which can be expressed as an IC50. The term “IC50,” as used herein, refers to the concentration of a drug, compound, molecule or carbamoyl ester that inhibits an activity or effect by 50%, e.g., by reducing binding of a competitor molecule to a protein (e.g., a receptor) by 50%; or by reducing the level of an activity (e.g., cholinesterase activity) by 50%.

As used herein, an “individual” is any mammal. A mammal can be a rodent (such as a rat, mouse or guinea pig), domesticated animal (such as a dog or cat), ruminant animal (such as a horse or a cow) or a primate (such as a monkey or a human). In a preferred embodiment, the individual is a human.

The carbamoyl esters of the invention can be employed in the methods, pharmaceutical compositions, kits and assays of the invention in a single dose or in multiple doses. The multiple doses can be administered as multiple doses in a single day, as a single daily dose administered for more than one day, as multiple doses administered daily for more than one day, or as a single dose on any given day followed or preceded by multiple doses in the intervening days. The multiple doses can be administered for a day, days, a week, weeks, a month, months, a year or years.

The carbamoyl esters of the invention can be administered to increase wakefulness to an individual acutely (briefly or short-term) or chronically (prolonged or long-term). For example, the carbamoyl esters of the invention can be used for wake promotion by administering the carbamoyl ester to the individual once a day, multiple times (e.g., 2, 3, 4) in a day, for a day, days, a week, weeks, a month, months or years. The carbamoyl ester of the invention can be administered as needed by the individual.

In one embodiment, the dose of the carbamoyl ester can be about 0.1 mg, about 1 mg, about 2.5 mg, about 5 mg, about 10 mg, about 15 mg, about 20 mg, about 25 mg, about 40 mg, about 50 mg, about 75 mg, about 90 mg, about 100 mg, about 150 mg, about 200 mg, about 250 mg, about 500 mg, about 750 mg or about 1000 mg.

In another embodiment, the dose of the carbamoyl ester can be between about 1 mg to about 100 mg; between about 2 mg to about 50 mg; or between about 5 mg to about 25 mg.

In still another embodiment, each dose of a multiple dose can be about 0.1 mg, about 1 mg, about 2.5 mg, about 5 mg, about 10 mg, about 20 mg, about 25 mg, about 40 mg, about 50 mg, about 75 mg, about 90 mg, about 100 mg, about 150 mg, about 200 mg, about 250 mg, about 500 mg, about 750 mg or about 1000 mg.

In a further embodiment, each dose of a multiple dose can be between about 1 mg to about 100 mg; between about 2 mg to about 50 mg; or between about 5 mg to about 25 mg.

The carbamoyl ester is administered for wake promotion or employed in the assays and kits of the invention in an effective amount. The term “effective amount,” “amount effective,” or “therapeutically effective amount,” when referring to the amount of the carbamoyl ester or pharmacologically active agent, is defined as that amount, or dose, of the carbamoyl ester or pharmacologically active agent that is sufficient for therapeutic efficacy (e.g., an amount sufficient to promote wakefulness; an amount sufficient to reduce drowsiness; an amount sufficient to increase mental alertness; an amount sufficient to prevent symptoms of MS fatigue; an amount sufficient to alleviate tiredness).

The carbamoyl ester can optionally be used for wake promotion, in kits and assays of the invention with an acceptable carrier. The selection of an acceptable carrier will depend upon the method, kit or assay. For example, an acceptable carrier in an in vitro method, assay or kit can be saline, a suitable buffer or cell culture media.

The carbamoyl esters of the invention can be administered alone or as admixtures with conventional excipients, for example, pharmaceutically, or physiologically, acceptable organic, or inorganic carrier substances suitable for enteral or parenteral application which do not deleteriously react with the compound employed in the method. Suitable pharmaceutically acceptable carriers include water, salt solutions (such as Ringer's solution), alcohols, oils, gelatins and carbohydrates such as lactose, amylose or starch, fatty acid esters, hydroxymethylcellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances which do not deleteriously react with the compounds employed in the methods of the invention. The preparations can also be combined, when desired, with other active substances to reduce metabolic degradation.

Methods of administration of the carbamoyl esters are oral administration (such as a tablet or capsule). The carbamoyl ester alone, or when combined with an admixture, can be administered in a single or in more than one dose over a period of time to confer the desired effect (e.g., to increase the duration of wakefulness).

The carbamoyl esters can be administered to a target site in an individual.

When parenteral application is needed or desired, particularly suitable admixtures for the carbamoyl esters are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The carbamoyl esters employed in the methods, assays or kits of the invention can also be incorporated into liposomes or administered by transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of which are hereby incorporated by reference.

The dosage and frequency (single or multiple doses) administered to an individual can vary depending upon a variety of factors, including, for example, the increase in duration of wakefulness needed, the pharmacologically active agent to be delivered; size, age, sex, health, body weight, body mass index and diet of the individual; nature and extent of wake promotion, kind of concurrent treatment, complications from the condition or impairment, or other health-related problems of the human being treated.

Other therapeutic regimens or agents can be used in conjunction with the carbamoyl esters of the invention employed for wake promotion. Adjustment and manipulation of established dosages (e.g., frequency and duration) are well within the ability of those skilled in the art.

The present invention is further illustrated by the following examples, which are not intended to be limiting in any way.

### Some Representative Compounds of the Invention are Shown in Table 1 Below:

TABLE 1

| | | Compound | |
|---|---|---|---|
| # | Structure | Name | |
| 9 | 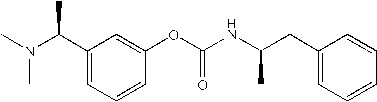 | S-riva-l-amphetamine | Sympathomimetic |
| 20 | 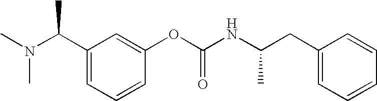 | S-riva-d-amphetamine | Sympathomimetic |
| 7 | 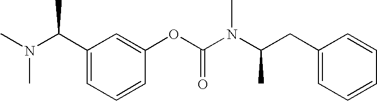 | S-riva-l-methamphetamine | Sympathomimetic |
| 13 | 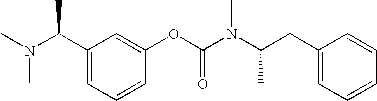 | S-riva-d-methamphetamine | Sympathomimetic |
| 14 | 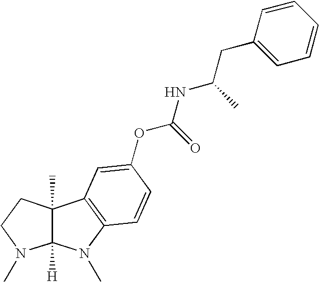 | Physo-d-amphetamine | Sympathomimetic |
| 15 | 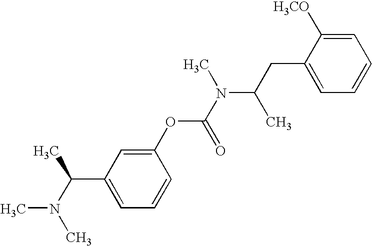 | S-riva-methoxyphenamine | Sympathomimetic |
| 16 | 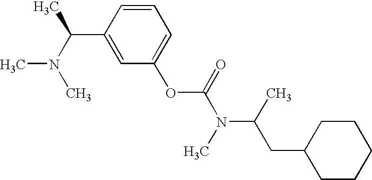 | S-riva-propylhexedrine | Sympathomimetic |
| 11 | 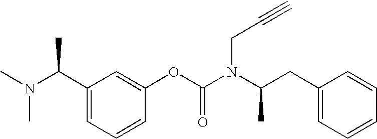 | S-riva-desmethylselegiline | Sympathomimetic |
| 17 | 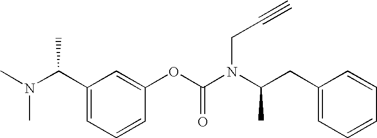 | R-riva-desmethylselegiline | Sympathomimetic |
| 18 | 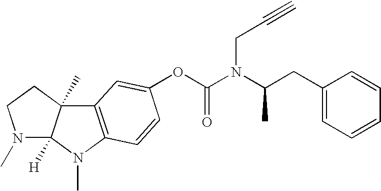 | Physo-desmethylselegiline | Sympathomimetic |
| 2 | 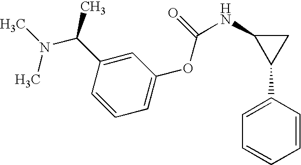 | S-riva-tranylcypromine | Sympathomimetic |
| 29 | 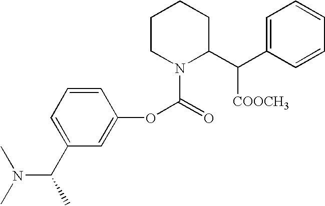 | S-riva-rac-methyiphenidate | Sympathomimetic |
| 5 | 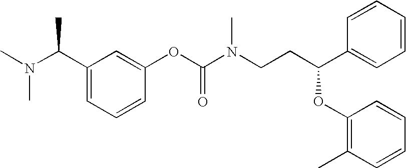 | S-riva-atomoxetine | Antidepressant |
| 3 | 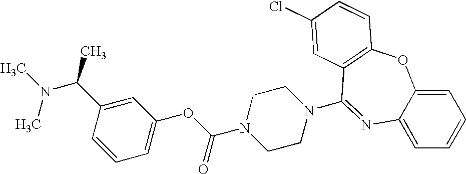 | S-riva-amoxapine | Antidepressant |
| 4 | 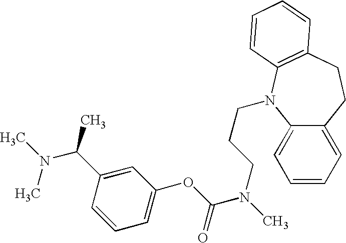 | S-riva-desipramine | Antidepressant |
| 5A | 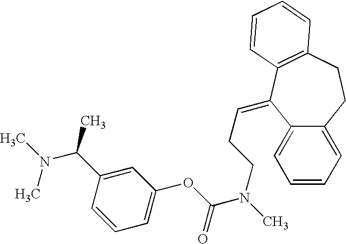 | S-riva-nortriptyline | Antidepressant |
| 6 | 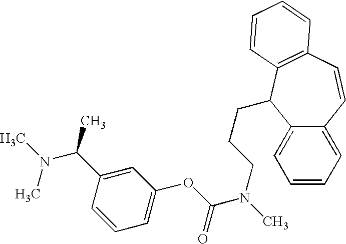 | S-riva-protriptyline | Antidepressant |
| 7A | 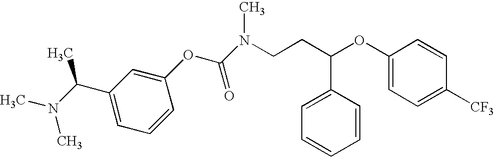 | S-riva-fluoxetine | Antidepressant |
| 8 | 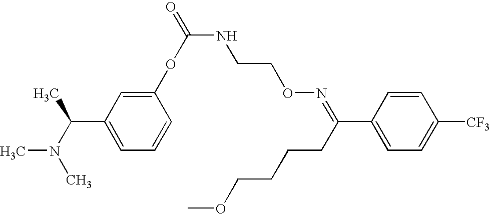 | S-riva-fluvoxamine | Antidepressant |
| 9A | 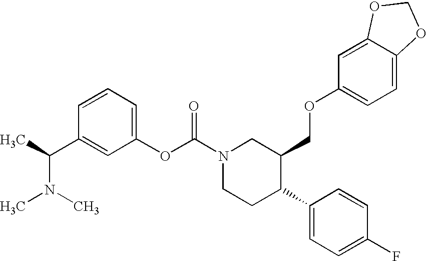 | S-riva-paroxetine | Antidepressant |
| 23 | 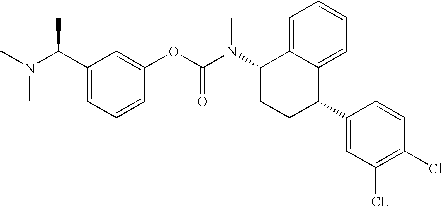 | S-riva-sertraline | Antidepressant |
| 10 | 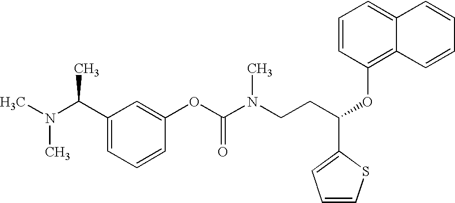 | S-riva-duloxetine | Antidepressant |

Further advantages and characteristics of the invention will be understood more clearly from the following examples. These examples, which in no way imply a limitation, are given by way of example.

### EXAMPLES

### Example 1

### Synthesis of S-Riva-Atomoxetine (5)

(S)-(−)-3′-hydroxyphenylethyldimethylamine (96 mg, 0.58 mmol) (1) was dissolved in 4 ml of dry ethyl acetate. N,N′-carbonyldiimidazole powder (283 mg, 1.74 mmol) was added and the mixture stirred at room temperature for 20 h. Acetic acid (313 mg, 5.22 mmol) was then added to the mixture, followed by the addition of 162 mg (−)-atomoxetine (4, 0.63 mmol). The resulting mixture was stirred at room temperature overnight. Saturated sodium bicarbonate solution was added to the mixture and the aqueous and organic layers separated. The aqueous layer was extracted twice with ethyl acetate. The organic layers were combined, dried over NaHCO₃, evaporated and purified with a silica gel column (eluted with 25% ethyl acetate in hexane with 1% triethylamine) to yield 101 mg of the carbamoyl ester (5) (0.23 mmol, 39.0% yield).

The carbamoyl ester (5) was confirmed by NMR. ¹H-NMR of the HCl salt (CDCl₃, 400 MHz): δ 1.808 and 1.825 (d, 3H, J=6.8 Hz, CH₃), 2.090-2.320 (m, 2H), 2.262 (ma) and 2.325 (mi) (s, 3H, CH₃), 2.506-2.541 (m, 3H, CH₃), 2.658-2.698 (m, 3H, CH₃), 3.002 (ma) and 3.082 (mi) (s, 3H, CH₃), 3.520-3.575 (m, 1H, CH), 3.662-3.700 and 3.892-3.961 (m, 1H, CH), 4.048-4.123 (m, 1H, CH), 5.180-5.252 (m, 1H, CH), 6.535-6.582 (m, 1H, CH arom.), 6.729-6.787 and 6.902-6.957 (m, 3H, 3×CH arom.), 7.007-7.086 (m, 2H, 2×CH arom.), 7.224-7.428 (m, 7H, 7×CH arom.), 12.620 (bs, 1H, HCl).

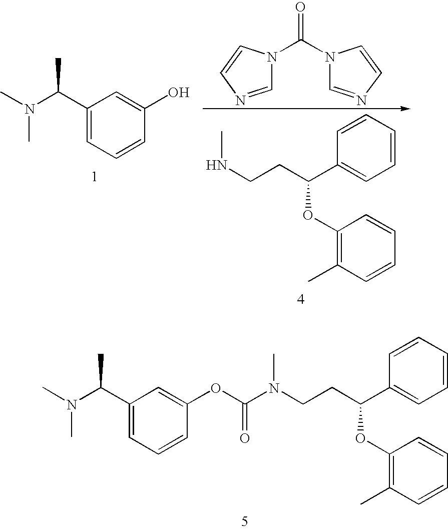

Free base 5 was converted into the hydrochloride salt following the procedure described below:

The carbamoyl ester (5) was dissolved in chloroform (3 mL per mmol free base 5). A solution of 1M HCl in ether (1.5-2 molar equivalents) was added dropwise at 0° C. Upon completion of addition of hydrochloric acid, the mixture was allowed to warm to room temperature. Solvents were removed by evaporation and the residue dried under vacuum to yield the hydrochloride salt of the carbamoyl ester (5) visible as a white to off-white solid.

### Example 2

### Synthesis of S-Riva-L-Methamphetamine (7)

4-nitrophenychloroformate powder (0.179 g, 0.86 mmol) was added to a solution of 0.12 g (0.72 mmol) (−)-3′-hydroxyphenylethyldimethylamine (1) and 0.22 g (2.17 mmol) triethylamine in 10 ml of dry dichloromethane (0.86 mmol) at 0° C. The solution was stirred at 0° C. for 5 min followed by stirring at room temperature for an additional 30 minutes. A solution of 0.107 g 1-methamphetamine (6) in 2 ml of dry dichloromethane was then added, and the resulting solution stirred at room temperature for 2 hours. The solvent was evaporated and the residue applied to a silica gel column. The compound (7) was eluted with 3% acetone in ethyl acetate containing 1% triethylamine. Fractions containing compound (7) were combined and concentrated to yield 0.15 g of the compound (7) (0.44 mmol, 61% yield).

The compound (7) was confirmed by NMR. ¹H-NMR (CDCl₃, 300 MHz): δ 1.192 (mi) and 1.275 (ma) (d, 3H, J=6.8 Hz, CH₃), 1.305 and 1.326 (d, 3H, J=3.0 Hz, CH₃), 2.162 and 2.167 (s, 6H, 2×CH₃), 2.746 (dd, 1H, J=13.7 and 6.8 Hz, CHH), 2.850 (dd, 1H, J=13.7 and 6.8 Hz, CHH), 2.868 and 2.886 (s, 3H, CH₃), 3.165-3.217 (m, 1H, CH), 4.558-4.633 (m, 1H, CH), 6.665 and 6.855 (bd, 1H, J=7.9 Hz, CH arom.), 6.723 and 6.928 (bs, 1H, CH arom.), 7.065 (bd, 1H, J=7.2 Hz, CH arom.), 7.176-7.305 (m, 6H, CH arom.).

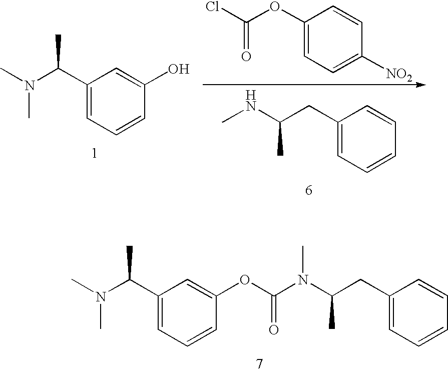

### Example 3A

### Synthesis of S-Riva-L-Amphetamine (9)

At room temperature, diisopropylethylamine (5.16 g, 40 mmol) and CDI powder (6.48 g, 40 mmol) were added to a suspension of 7.34 g of 1-amphetamine sulfate (8) (40 mmol) in 140 ml of dichloromethane. The resulting mixture was stirred at room temperature for 1 h. (−)-3′-hydroxyphenylethyldimethylamine (1) (3.3 g, 20 mmol), which had been mixed with 0.8 g sodium hydride (60% dispersion in mineral oil) in dry toluene (120 ml) for 30 minutes, was added to the mixture and the dichloromethane removed under reduced pressure. The resulting suspension was heated to 85° C. overnight with stirring. The reaction mixture was extracted with 0.5 M HCl (200 ml). The aqueous layer was washed with ethyl acetate, basified at 0° C. to pH ˜11 with sodium bicarbonate and 0.5 N NaOH and extracted with ethyl acetate (3×100 ml). The organic layers were combined, dried over sodium sulfate and evaporated. The residue was purified with a silica gel column. Elution with a mixture of 20-30% ethyl acetate with 1% triethylamine in hexane yielded 1.53 g of the carbamoyl ester (9) (4.7 mmol, 23.5% yield).

The carbamoyl ester (9) was confirmed by NMR. ¹H-NMR (CDCl₃, 300 MHz): δ 1.179 (d, 3H, J=6.6 Hz, CH₃), 1.331 (d, 3H, J=6.7 Hz, CH₃), 2.174 (s, 6H, 2×CH₃), 2.789 (dd, 1H, J=13.4 and 7.2 Hz, CHH), 2.832 (dd, 1H, J=13.4 and 5.9 Hz, CHH), 3.228 (q, 1H, J=6.7 Hz, CH), 3.980-4.062 (m, 1H, CH), 4.856 (bd, 1H, J=7.2 Hz, NH), 6.955 (bd, 1H, J=7.4 Hz, CH arom.), 7.018 (bs, 1H, CH arom.), 7.095 (bd, 1H, J=7.7 Hz, CH arom.) 7.186-7.303 (m, 6H, CH arom.).

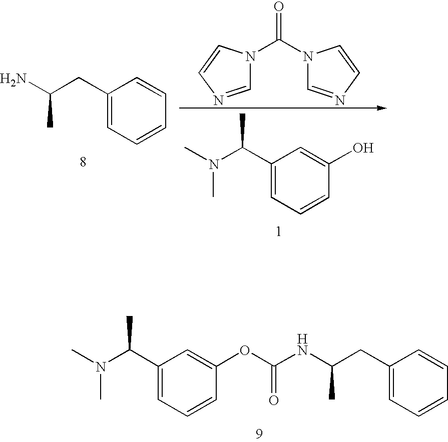

### Example 3B

### Alternative Synthesis of S-Riva-L-Amphetamine (9)

(S)-(−)-3′-hydroxyphenylethyldimethylamine (1) (1.2 g, 7.3 mmol) was dissolved in 20 ml of dry ethyl acetate. N,N′-carbonyldiimidazole powder (2.37 g, 14.6 mmol) was added and the mixture stirred at 85° C. overnight. After cooling to 0° C., 3.3 g of acetic acid (55.0 mmol) was added, followed by the addition of 2.8 g of 1-amphetamine (8) (20.7 mmol). The mixture was stirred at room temperature for 36 h. Water (20 ml) and 1M HCl (20 ml) were added and the aqueous and organic layers separated. The organic layer was extracted with 0.5M HCl. The aqueous layers were combined and washed with ether twice, basified with NaHCO₃ and 0.5 N NaOH to pH ˜11 and extracted with ether. The ether layer was dried over NaHCO₃, evaporated and purified with silica gel chromatography. Elution with a mixture of 25% ethyl acetate with 1% triethylamine in hexane yielded 0.93 g of the carbamoyl ester (9) (2.85 mmol, 39% yield).

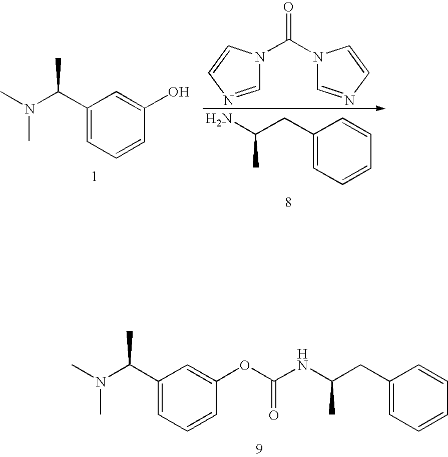

### Example 4

### Synthesis of S-Riva-Desmethylselegiline (11)

Triphosgene (85.5 mg, 0.28 mmol) was dissolved in 2 ml of dry dichloromethane. To this solution, a mixture of 145 mg of desmethylselegiline (10) (0.84 mmol) and 110 mg of diisopropylethylamine (DIEA) (0.85 mol) in 1 ml of dry dichloromethane was added at 0° C. and allowed to react for 10 minutes. The mixture was stirred at room temperature for 60 hours, and subsequently added to a suspension of (−)-_-3′-hydroxyphenylethyldimethylamine (1) (92 mg, 0.55 mmol) and sodium hydride (68 mg, 60% dispersion in mineral oil) in dry acetonitrile, which had been stirred at room temperature for 1 hour. The resulting mixture was stirred at room temperature overnight. The solvents of the above mixture were removed under reduced pressure. The residue was dissolved in 0.5 M HCl and washed with ether. The aqueous layer was basified with sodium bicarbonate and extracted with ethyl acetate (3×20 ml). The organic layer was washed with 0.5 N NaOH (200 ml), dried over sodium sulfate and evaporated. The residue was purified with a silica gel column (eluted with 30-60% ethyl acetate in hexane with 1% triethylamine) to yield 185 mg of the carbamoyl ester (11) (0.508 mmol, 92.3% yield).

The carbamoyl ester (11) was confirmed by NMR. ¹H-NMR (CDCl₃, 300 MHz): δ 1.339 (d, 3H, J=6.6 Hz, CH₃), 1.327-1.415 (m, 3H, CH₃), 2.187 (s, 6H, 2×CH₃), 2.215-2.258 (m, 1H, CH), 2.843-2.870 (m, 1H, CH), 3.063 (dd, 1H, J=13.5 and 7.5 Hz, CHH), 3.230 (q, 1H, J=6.6 Hz, CH), 4.043-4.118 (m, 2H, 2×CH), 4.372-4.411 (m, 1H, CH), 6.846-7.024 (m, 2H, 2×CH arom.), 7.108 (bd, 1H, J=7.7 Hz, CH arom.), 7.202-7.313 (m, 6H, CH arom.).

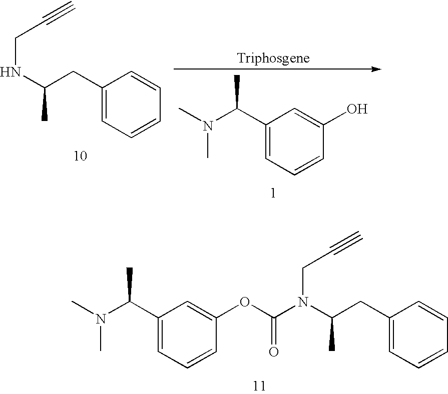

### Example 5

### Synthesis of S-Riva-D-Amphetamine (20)

(S)-(−)-3′-hydroxyphenylethyldimethylamine (1) (81 mg, 0.49 mmol) was dissolved in 4 ml of dry ethyl acetate. N,N′-carbonyldiimidazole powder (199 mg, 1.23 mmol) was added and the mixture was stirred at room temperature for 20 h. Acetic acid (184 mg, 3.07 mmol) was added, followed by the addition of 186 mg of d-amphetamine (19) acetate salt (0.96 mmol). The mixture was stirred at room temperature overnight. Water (5 ml) and 1M HCl (5 ml) were added and the aqueous and organic layers separated. The organic layer was extracted with 0.5M HCl. The aqueous layers were combined, washed with ether twice and basified with NaHCO₃ and 0.5 N NaOH to pH ˜11, followed by extraction with ether. The ether layer was dried over NaHCO₃, evaporated and purified with a silica gel column (eluted with 25% ethyl acetate in hexane with 1% triethylamine) to yield 95 mg of carbamoyl ester (20) (0.29 mmol, 59.4% yield).

The carbamoyl ester (20) was confirmed by NMR. ¹H-NMR (CDCl₃, 300 MHz): δ 1.192 (d, 3H, J=6.6 Hz, CH₃), 1.367 (d, 3H, J=6.7 Hz, CH₃), 2.205 (s, 6H, 2×CH₃), 2.759 (dd, 1H, J=13.4 and 7.2 Hz, CHH), 2.896 (dd, 1H, J=13.4 and 5.9 Hz, CHH), 3.295 (q, 1H, J=6.6 Hz, CH), 3.990-4.044 (m, 1H, CH), 4.847 (bd, 1H, J=7.2 Hz, NH), 6.966 (bd, 1H, J=7.4 Hz, CH arom.), 6.976 (bs, 1H, CH arom.), 7.114 (bd, 1H, J=7.7 Hz, CH arom.) 7.191-7.324 (m, 6H, CH arom.).

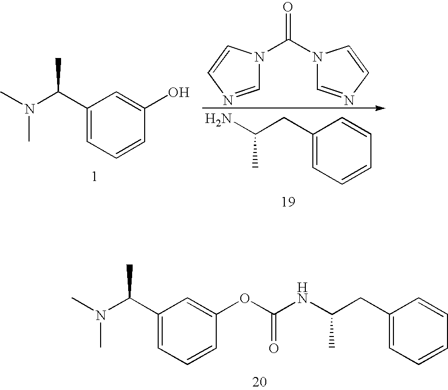

### Example 6

### Compound Synthesis

Compounds of the invention are produced by coupling of Rₐ-phenol and Q-H using methods known to those skilled in the art. For example,

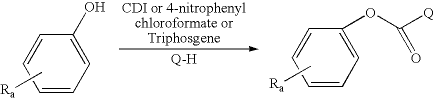

wherein Rₐ represents the appropriate phenyl substituents for a stigmine, such as rivastigmine or physostigmine, and Q represents an amine-containing pharmacologically active agent. For example,

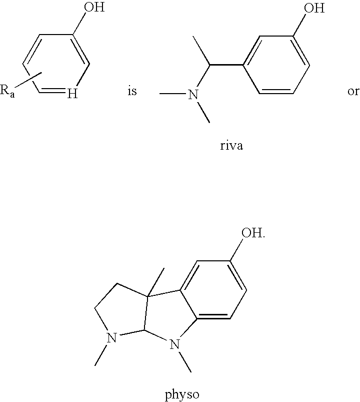

Exemplary compounds are shown in the table below.

| Starting material | Reagents/conditions | Results |
|---|---|---|
| Desipramine (300 | Desipramine is treated with sodium | 4 (240 mg, 52% yield, >95% |
| mg, 1.0 mmol) | bicarbonate and riva carbamate | by HPLC.) isolated by |
| | imidazole solution (2.0 mmol, 2.0 eq.) | column chromatography. |
| | in dichloromethane (8 mL). | |
| Fluvoxamine maleate | Fluvoxamine is treated with sodium | 8 (10 mg, 8% yield, 90% |
| (100 mg, 0.23 mmol) | bicarbonate and riva carbamate | purity by HPLC) isolated by |
| R₁ = H | imidazole solution (0.66 mmol, 3.0 eq.) | preparative TLC. |
| | in dichloromethane (7 mL). | |
| Fluoxetine | Fluoxetine is treated with | 7A isolated by preparative |
| hydrochloride (100 | diisopropylethylamine (0.63 mmol, 2.2 | TLC to give 30 mg, 20% |
| mg, 0.29 mmol) | eq.) and riva carbamate imidazole | yield, 80% purity by HPLC. |
| | solution (0.63 mmol, 2.2 eq) in | |
| | dichloromethane (6 mL). | |
| Paroxetine (87 mg, | Riva carbamate soln in dichloromethane | 9A (49 mg, 83% purity). |
| 0.26 mmol) | (S-rivastigmine coupled with | |
| | carbonyldiimidazole) 1.2 mmol., | |
| | dichloromethane (4 mL) | |
| Sertraline maleate | Sertraline is treated with sodium | 23 |
| (250 mg, 0.73 mmol) | bicarbonate and riva carbamate | |
| | imidazole solution (1.5 mmol, 2.05 eq.) | |
| | in the presence of | |
| | diisopropylethylamine (2.87 mmol, 3.9 | |
| | eq.) in dichloromethane (15 mL). | |
| Methylphenidate HCl | 1)Methylphenidate is treated with 2.0 M | 29 |
| (270 mg, 1.0 mmol) | aq. soln. of Na₂CO₃, dried and | |
| | concentrated, riva carbamate soln (2.4 | |
| | mL of 0.25 M soln in dichloromethane), | |
| | dichloromethane (2 mL) | |
| | 2)diisopropylethylamine (130 mg, 1.0 | |
| | mmol) added and stirred | |
| Protriptyline HCl (2 | Carbonyldiimidazole (6.67 mmol), (S)- | 6 Purified twice on silica |
| g, 6.67 mmol) | rivastigmine phenol (6.67 mmol), | column chromatography to |
| | diisopropylethylamine (10.0 mmol), | give 1.15 g of the desired |
| | dichloromethane (60 mL) | product (HPLC purity >99%). |
| Fluoxetine HCl (2 g, | Carbonyldiimidazole (6 mmol), (S)- | 7A Purified on a silica |
| 6 mmol) | rivastigmine phenol (6 mmol), | column chromatography to |
| | diisopropylethylamine (9 mmol), | give 1.05 g of the desired |
| | dichloromethane (40 mL) | product (HPLC purity >99%) |
| Duloxetine (740 mg, | Carbonyldiimidazole (2.6 mmol), (S)- | 10 |
| 2.5 mmol) | rivastigmine phenol (2.7 mmol), | |
| | dichloromethane (10 mL) | |
| Fluvoxamine maleate | Carbonyldiimidazole (1.05 mmol), (S)- | 8 LC/MS of the reaction |
| (434 mg, 1 mmol) | rivastigmine phenol (1.1 mmol), | mixture showed the mass of |
| | diisopropylethylamine (3 mmol), | the product. |
| | dichloromethane (6 mL) | |
| Fluvoxamine maleate | Carbonyldiimidazole (6.05 mmol), (S)- | 8 LC/MS of the reaction |
| (2.5 g, 5.7 mmol) | rivastigmine phenol (6.3 mmol), | mixture showed the mass of |
| | diisopropylethylamine (17.3 mmol), | the product. |
| | dichloromethane (40 mL) | |

### Example 7

### Purification of Compounds of the Invention from S-Rivastigmine and 1-Amphetamine

Samples of the carbamoyl ester obtained from S-rivastigmine and 1-amphetamine were dissolved in water (30 mL) and adjusted to a pH of 10 using 2.0 M aq. solution of Na₂CO₃. The carbamoyl ester free base was then extracted with dichloromethane (2×30 mL), dried (Na₂SO₄) and concentrated using a rotovap. The residue was passed through a silica column using heptanes (74%), ethyl acetate (25%) and triethylamine (1%) as the solvent. The fractions were evaporated using a rotovap and dried under high vacuum overnight. The residue was taken up in water (6 mL), followed by the addition of 2.0 M HCl (3 mL) gave a clear homogeneous solution. It was then lyophilized to give the carbamoyl ester HCl (278 mg, HPLC purity >99%). The carbamoyl ester is shown below:

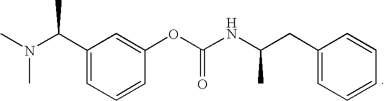

The lyophilized material was a white, free flowing powder where as the sample before purification and lyophilization was sticky and was hard to transfer.

### Examples 8A and 8B

### Preparation of Hydrochloride Salts of Compounds of the Invention

### Example 8A

A compound of the invention is dissolved in chloroform (3 ml per mmol compound). A solution of 1M HCl in ether (1.5-2 molar equivalents) is added dropwise at 0° C. Upon completion of addition of hydrochloric acid, the mixture is allowed to warm to room temperature. Solvents are removed by evaporation and the residue dried under vacuum to yield the hydrochloride salt of the compound.

### Example 8B

A compound is dissolved in water and adjusted to a pH of ˜10 using 2.0 M aq. solution of Na₂CO₃. The compound is then extracted with dichloromethane (2×30 mL), dried (Na₂SO₄) and concentrated. The residue is passed through a silica column using heptanes (74%), ethyl acetate (25%) and triethylamine (1%) as the solvent. The fractions are evaporated using a rotovap and dried under high vacuum overnight. The residue is taken up in water (6 mL), followed by the addition of 2.0 M HCl (3 mL). The solution is then lyophilized to give the compound as its HCl salt.

### Example 9

### Compounds Inhibit Acetylcholinesterase In Vitro

All reagents employed in this experiment were of analytical grade. Acetylthiocholine iodide and 5,5′-dithiobis-(2-nitro)benzoic acid (DTNB) and human recombinant acetylcholinesterase (C1682) were purchased from Sigma Chemical Co (St. Louis, Mo.).

Acetylcholinesterase activity of compounds was determined at 25° C. by a modification of the colorimetric method of Ellmann, et al. (Biochem. Pharmacol., 7:88-95 (1961)). The enzyme, compound or stigmine and buffer were preincubated for 30 minutes. At the end of the preincubation period, the substrate acetylthiocholine was added. The final assay mixture contained 10 mM Tris-buffer (pH 8), 0.3 mM Acetylthiocholine and 0.33 mM DTNB and 0.08 U/ml enzyme. At least five (5) different concentrations of the compound or stigmine were assayed per IC50 experiment.

Hydrolysis of acetylthiocholine was monitored indirectly by measurement of the formation of the conjugate between thiocholine and DTNB. Optical density at 405 nm was recorded during 5 minutes employing a microplate spectrophotometer (Polarstar, BMG Labtech) and plotted against time. The inverse of the initial rates for a range of inhibitor concentrations was plotted against concentration (Dixon Plot) to give the IC50 value (the concentration at which enzyme activity is inhibited by 50%) as the opposite value of the x-intercept (Burlingham, et al., J. Chem. Ed., 80:214-218 (2003)).

The inhibition of butylryl cholinesterase was determined using methods known in the art e.g., Alcala Molel M. et al. (2003) Characterization of the anticholinesterase activity of two new tacrin-huperzine A hybrids. Neuropharmacology, 44 (6), 749-755.

The results are summarized as follows:

| | | Inhibition | Inhibition |
|---|---|---|---|
| Compound | | of AChE (%) | of BuChE (%) |
| No. | Name | 10 μM | 10 μM |
| | S-rivastigmine | 19 | 100 |
| 9 | S-riva-l-amphetamine | 79 | 90 |
| 20 | S-riva-d-amphetamine | 76 | 20 |
| 7 | S-riva-l-methamphetamine | 79 | 17 |
| 13 | S-riva-d-methamphetamine | 97 | 95 |
| 14 | Physo-d-amphetamine | 94 | 28 |
| 15 | S-riva-methoxyphenamine | 95 | 30 |

| Compound | | Inhibition of AChE |
|---|---|---|
| No. | Name | IC50 (μM) |
| | S-rivastigmine | 35.5 |
| 9 | S-riva-l-amphetamine | 3.1 |
| 20 | S-riva-d-amphetamine | 3.1 |
| 7 | S-riva-l-methamphetamine | 3.2 |
| 13 | S-riva-d-methamphetamine | 1.1 |
| 15 | S-riva-methoxyphenamine | 1.1 |

| | | | AChE |
|---|---|---|---|
| Cmpd # | Name | Compound Structure | IC50 (μM) |
| 1 | S-rivastigmine | 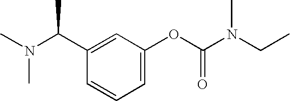 | 35.5 |
| 1A | Physostigmine | 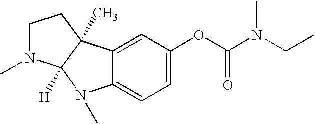 | 0.07 |
| 2 | S-rivatranylcypromine | 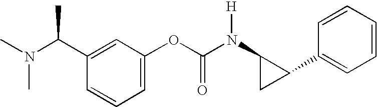 | 0.2 |
| 3 | S-riva-amoxapine | 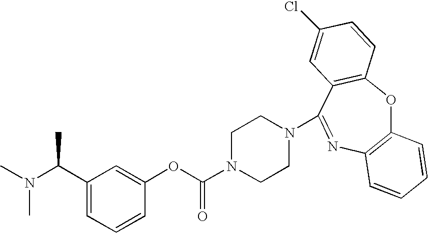 | 20.9 |
| 4 | S-riva-desipramine | 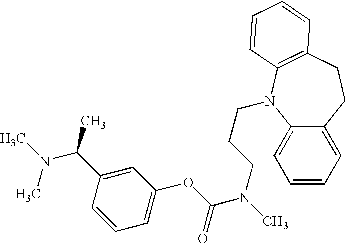 | 0.2 |
| 5A | S-riva-nortriptyline | 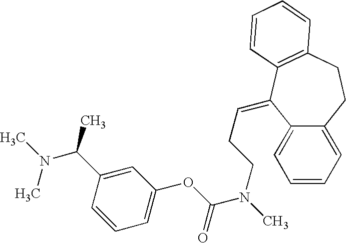 | 0.5 |
| 6 | S-riva-protriptyline | 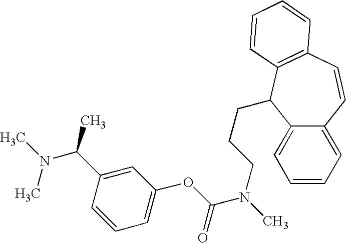 | 0.5 |
| 7A | S-rivafluoxetine | 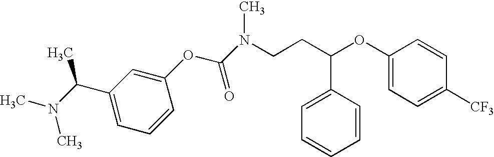 | 4.8 |
| 8 | S-rivafluvoxamine | 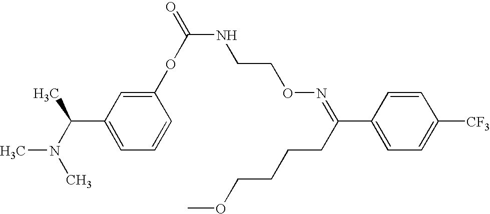 | 6.1 |
| 9A | S-rivaparoxetine | 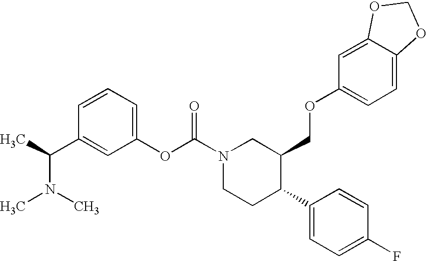 | 9.5 |
| 10 | S-rivaduloxetine | 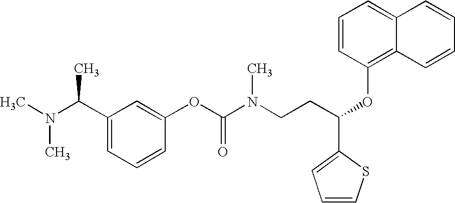 | 0.2-0.5 |
| 16 | S-rivapropylhexedrine | 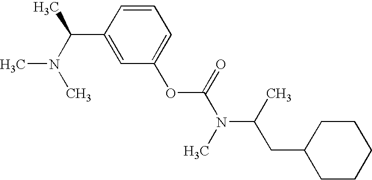 | 0.9 |

These data show that the compounds of the invention inhibit acetylcholinesterase in vitro. Inhibition of acetylcholinesterase by carbamoyl esters can be greater than inhibition of acetylcholinesterase by a stigmine, such as rivastigmine. Carbamoyl esters synthesized from stigmines resulted in similar or increased activity compared to the stigmine. For example, the carbamoyl ester (14) resulted in a 10-fold increase in enzymatic activity compared to rivastigmine. Thus, structural alterations in stigmines, carbamoyl esters with known enzymatic activity, did not decrease the enzyme inhibitory activity of the stigmine. Conversely, inhibition of butyrylcholinesterase by carbamoyl esters may be lower than inhibition of butyrylcholinesterase by a stigmine, such as rivastigmine. This shift towards acetyl over butyryl-cholinesterase inhibition is an unanticipated advantage of carbamoyl esters. Since acetylcholinesterase is relatively more highly expressed in the CNS than butyrylcholinesterase, whereas butyrylcholinesterase is more highly expressed in peripheral tissues than acetylcholinesterase, the carbamoyl esters are more selective for their intended target enzyme in the CNS than are stigmines, and hence are expected to have an improved efficacy versus tolerability ratio.

### Example 10

### Compounds of the Invention Inhibit Cholinesterase in Brain

Male Wistar rats were injected intraperitoneally (i.p.) with rivastigmine or with compounds of the invention. The dose of rivastigmine or carbamoyl ester resulted in a cholinergic behavioral effect with minimal side effects and was well-tolerated by the animals. Animals were decapitated 3 hours after injection and the brains rapidly removed. The brain tissue was diced into small pieces, placed on ice and immediately homogenized with a Polytron PT1200 (Kinematic AG) in 10 ml ice cold Tris with 0.1% Triton-X and protease inhibitors. The protease inhibitors in the extraction buffer were Antipain (10 μM), Aprotinin (5 TIU/mg protein), Bestatin (60 nm), Leupeptin (10 μM) and Pepstatin (1 μM). The final dilution of the homogenate in the final assay mixture was 120-fold.

Total cholinesterase activity was determined by a modification of the colorimetric method of Ellmann, et al. (Biochem. Pharmacol., 7:88-95 (1961)), as described above. Hydrolysis of acetylthiocholine was monitored indirectly by measurement of the formation of the conjugate between thiocholine and DTNB. Optical density at 405 nm was recorded during five (5) minutes employing a microplate spectrophotometer (Polarstar, BMG Labtech), and plotted against time. The initial rates were calculated from the slope of the linear portion of the graph.

Cholinesterase activity was normalized for protein content of the homogenate. Relative cholinesterase activity was calculated as the ratio of normalized cholinesterase activity in a rat treated with a control compound or a carbamoyl ester over normalized cholinesterase activity in saline treated rats.

These data are summarized below:

| | | Relative ChE | |
|---|---|---|---|
| Compound | dose | Activity | ChE inhibition |
| Rivastigmine | 2 mg/kg | 85% | 15% |
| 7 | 2 mg/kg | 62% | 38% |
| 9 | 8 mg/kg | 59% | 41% |

These data show that systemic administration of compounds of the invention results in inhibition of total cholinesterase activity in the brain of mammals. The carbamoyl esters resulted in significantly increased inhibition of cholinesterase activity in the brain compared to rivastigmine with minimal side effects. Thus, the carbamoyl esters of the invention can be employed in methods that inhibit cholinesterases with few side effects compared to currently available cholinesterase inhibitors.

### Example 11

### In Vitro Screening of Compounds of the Invention

An in vitro screening assay with various conjugates was completed according to the methods described in Ellman G L et al., Biochem Pharmacol., 7:88-95 (1961) and in Nadarajah B, J. Anal. Toxicol., 16:192-193 (1992), both of which are herein incorporated by reference in their entireties. The assay method was completed according to the following:

| | |
|---|---|
| Source | Human recombinant HEK-293 cells |
| Substrate | 700 μM acetylthiocholine |
| Vehicle | 1% DMSO |
| Pre-Incubation Time/Temp. | 15 minutes at 25° C. |
| Incubation Time/Temp. | 20 minutes at 25° C. |
| Incubation Buffer | 0.1 M sodium phosphate, pH 7.4 |
| Quantitation Method | Spectrophotometric quantitation of |
| | thiocholine |
| Significance Criteria | ≧50% of max stimulation or inhibition |

The assay results are summarized below:

| | | | AChEI | BuChEI | BuChEI |
|---|---|---|---|---|---|
| | Cmpd | AChEI (%) | (%) | (%) | (%) |
| Compound | No. | 10 μM | 1 μM | 10 μM | 1 μM |
| S-rivastigmine | | 19 | | 100 | 45 |
| Phenserine | | 99 | 93 | 42 | |
| S-rivastigmine- | 9 | 79 | 35 | 90 | 27 |
| 1-amphetamine | | | | | |
| S-rivastigmine-d- | 20 | 76 | 35 | 20 | |
| amphetamine | | | | | |
| S-rivastigmine-l- | 7 | 79 | 32 | 17 | |
| methamphetamine | | | | | |
| S-rivastigmine-d- | 13 | 97 | 51 | 95 | 25 |
| methamphetamine | | | | | |
| Physostigmine-d- | 14 | 94 | 45 | 28 | |
| amphetamine | | | | | |
| S-rivastigmine- | 15 | 95 | 49 | 30 | |
| methoxyphenamine | | | | | |
| S-rivastigmine- | 11 | 52 | 13 | 17 | |
| desmethylselegiline | | | | | |
| R-rivastigmine- | 17 | 31 | | 12 | |
| desmethylselegiline | | | | | |
| Physostigmine- | 18 | 37 | | 8 | |
| desmethylselegiline | | | | | |
| S-rivastigmine- | 2 | 97 | 84 | 67 | 23 |
| tranylcypromine | | | | | |
| S-rivastigmine- | 5 | 89 | 29 | 14 | |
| atomoxetine | | | | | |
| S-rivastigmine- | 3 | 16 | | 6 | |
| amoxapine | | | | | |
| S-rivastigmine- | 4 | 99 | 91 | 60 | 16 |
| desipramine | | | | | |
| S-rivastigmine- | 5A | 99 | 81 | 55 | 24 |
| nortriptyline | | | | | |
| S-rivastigmine- | 6 | 99 | 81 | 53 | 16 |
| protriptyline | | | | | |
| S-rivastigmine- | 7A | 76 | 11 | 15 | |
| fluoxetine | | | | | |
| S-rivastigmine- | 8 | 76 | 17 | 6 | |
| fluvoxamine | | | | | |
| S-rivastigmine- | 9A | 69 | 9 | 4 | |
| paroxetine | | | | | |
| S-rivastigmine- | 10 | 100 | 88 | 36 | |
| duloxetine | | | | | |

### Example 13

### Induction of Hypothermia by Carbamoyl Esters

Cholinergic agonists cause hypothermia through a central mechanism of action (Freedman et al, 1989. Direct measurement of muscarinic agents in the central nervous system of mice using ex vivo binding. Eur. J. Pharmacol. 174:253-260). Hypothermia can be used a method to determine centrally active doses and duration of action of acetylcholinesterase inhibitors in vivo (Rupniak et al, 1992. Reversal of cognitive impairment by heptyl physostigmine, a long-lasting cholinesterase inhibitor, in primates. J. Neurol. Sci. 107:246-249). Since a premise of the present invention is that carbomyl esters would liberate the conjugated amine in the CNS only after inhibition of acetylcholinesterase, induction of hypothermia provides a useful correlation with the acetylcholinesterase inhibition detected in vitro and in vivo.

Like rivastigmine, subcutaneous administration of carbamoyl esters (Compounds A, B, C and D) caused a dose-related-related fall in body temperature. Hypothermia was defined as a fall in temperature of >0.5° C. Table A shows the minimum effective dose (MED) for each compound.

TABLE A
Induction of hypothermia by carbamoyl esters

| Compound # | Compound Name/Letter Code | MED (mg/kg s.c.) |
|---|---|---|
| 1 | s-Rivastigmine | ≦0.1 |
| 9 | s-Riva-l-amphetamine/A | 10 |
| 7 | s-Riva-l-methamphetamine/B | ≦1 |
| 20 | s-Riva-d-amphetamine/C | ≦1 |
| 13 | s-Riva-d-methamphetamine/D | 10 |

### Example 13

### Measurement of Hypothermia and Determination of the Dose Range and Time Course for Cholinergic Effects

The induction of hypothermia was determined for compounds of the invention according to the methods described in Freedman, et al., European Journal of Pharmacology, 187 (1990), 193-199, which is incorporated by reference herein. Hypothermia is a marker of CNS penetration for ACHE inhibitors.

The dose range and time course for cholinergic effects of the compounds of the invention was determined as described below.

Subjects: Two hundred eight male CD IGS (Sprague Dawley derived) rats were received at 126-150 grams and maintained four per cage on a regular light/dark cycle (lights on 0600-1800) with ad libitum food and water for about 1 week before commencement of experimentation.
Apparatus: Injection was done with a 25-gauge ⅝-inch needle on a 1-mL tuberculin syringe. Observation was done in a 5½-×10-inch polycarbonate rat housing cage. Temperature was taken with a rat rectal probe on a Model BAT-12 electronic thermometer.
Compound Preparation: Test compounds were dissolved for example, in 0.9% saline. Concentrations for lower doses were prepared by taking aliquots from higher concentrations and diluting. Injection volumes were 1 mL/kg, if the test compound was sufficiently soluble. If less soluble, the maximum injection volume was 5 mL/kg. Route of administration was s.c.
A sample protocol is as follows:
Treatment Groups included (N=3, with 6 for Saline)

### Saline

(S)-Rivastigmine at 1, 3, 10, 30, and 100 mg/kg
(R)-Rivastigmine at 1, 3, 10, 30, and 100 mg/kg
Test Compounds were dosed at 1, 3, 10, 30, and 100 mg/kg
Procedure: The rats were brought to the test room in the home cage. Baseline temperatures were taken just before injection. After injection s.c., the rat was placed in the observation cage. At 0.5, 1, 2, and 4 hours after injection, it was observed briefly for gross signs; salivation was scored as absent, clearly present, or copious; and rectal temperature was taken. Salivation score and temperature were determined within this constraint, but only the most salient of gross signs were noted. After the 4-hour observation point, or sooner if signs of distress were observed, the rat was euthanized by CO₂ inhalation.
Data Analysis: Gross signs, salivation score, and temperature at each time point were tabulated for inspection.
Results of the hypothermia and dose determination are shown below.

| | | | Hypothermia | Max | |
|---|---|---|---|---|---|
| Cmpd | | | MED | Tolerated | TI |
| # | Name | Compound Structure | (mg/kg) | Dose | MTD/MED |
| 1 | S-rivastigmine | 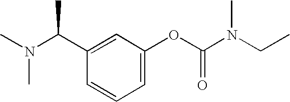 | ≦0.1 | 10 | ≧100 |
| 3 | S-riva-amoxapine | 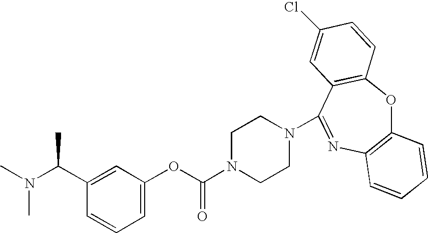 | 10 | ≧100 | ≧10 |
| 4 | S-riva-desipramine | 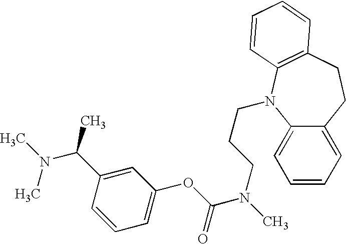 | 3 | ≧100 | ≧30 |
| 5 | S-riva-nortriptyline | 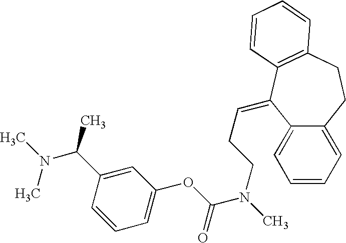 | ≦1 | ≧100 | ≧100 |
| 6 | S-riva-protriptyline | 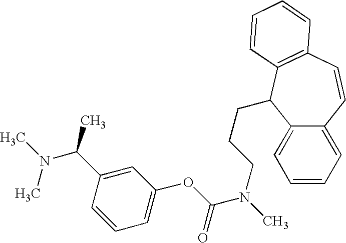 | 100 | >100 | >1 |
| 2 | S-riva-tranyl-cypromine | 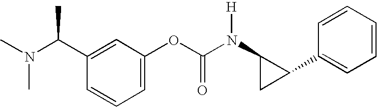 | 10 | 30 | 3 |
| 7A | S-riva-fluoxetine | 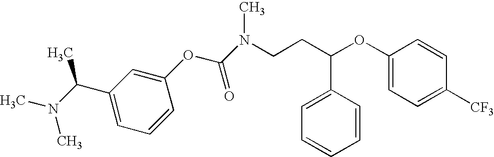 | 3 | ≧100 | ≧30 |
| 16 | S-riva-propyl-hexedrine | 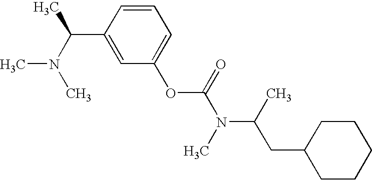 | 10 | ≧100 | ≧10 |

### Example 18

### Wake Promotion by Carbamoyl Esters

Certain clinical conditions are characterized by unpredictable bouts of sleepiness that can interfere with the ability to conduct activities of daily living, such as driving. Examples are narcolepsy, and disturbances of diurnal rhythm, such as adjustment to shift work. Currently approved therapies for such conditions are amphetamines and modifenil. Significant limitations of available therapies include rebound hypersomnolence and abuse potential.

Test compounds in various dose ranges or vehicle were administered to male Wistar rats 5 hours after lights on (CT-5). EEG, EMG, locomotor activity, drink- and food-related activity, and body temperature were concurrently monitored for 30 hr before and after treatment from rats living in separate isolated recording chambers. Sleep-wake discriminations were carried out using SCORE2004™, proprietary real-time hardware and software technology of Hypnion, Inc. (Lexington, Mass.). Comparisons were made between the reference compounds: d-amphetamine, rivastigmine, modafinil, and the following carbamoyl ester test compounds:

| Compound # | Compound Name/Letter Code |
|---|---|
| 9 | s-riva-l-amphetamine/A |
| 7 | s-riva-l-methamphetamine/B |
| 20 | s-riva-d-amphetamine/C |
| 13 | s-riva-d-methamphetamine/D |

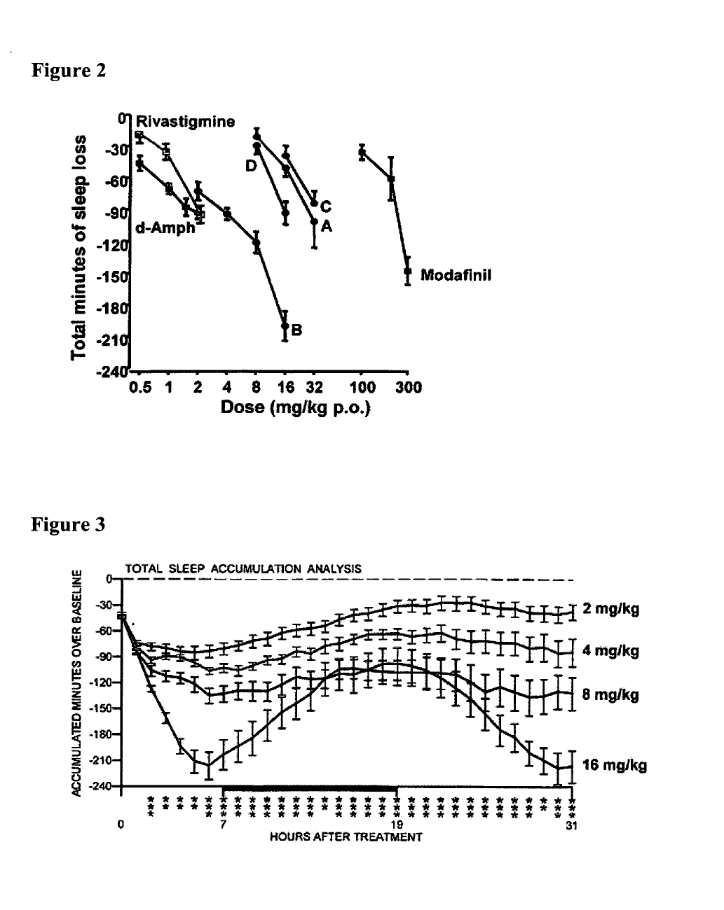

Administration of d-amphetamine or modafinil increased the duration of wakefulness (i.e., increased the total number of minutes of sleep loss) in a dose-dependent manner. Although not approved as a wake promoting agent, rivastigmine also increased wakefulness (FIG. 2). Higher doses of reference compounds were not tested because of tolerability. Similarly, the carbamoyl esters A, B, C and D caused a dose-related increase in wakefulness. Of these, Compound B caused an unexpectedly long increase in wakefulness that surpassed that seen with the reference compounds tested (FIG. 2).

Unlike rivastigmine, rebound hypersomnolence was not observed following administration of the carbamoyl ester, Compound B (FIG. 3). This is an unexpected finding that would not be predicted from the known actions of the carbamoyl ester's component stigmine or amine.

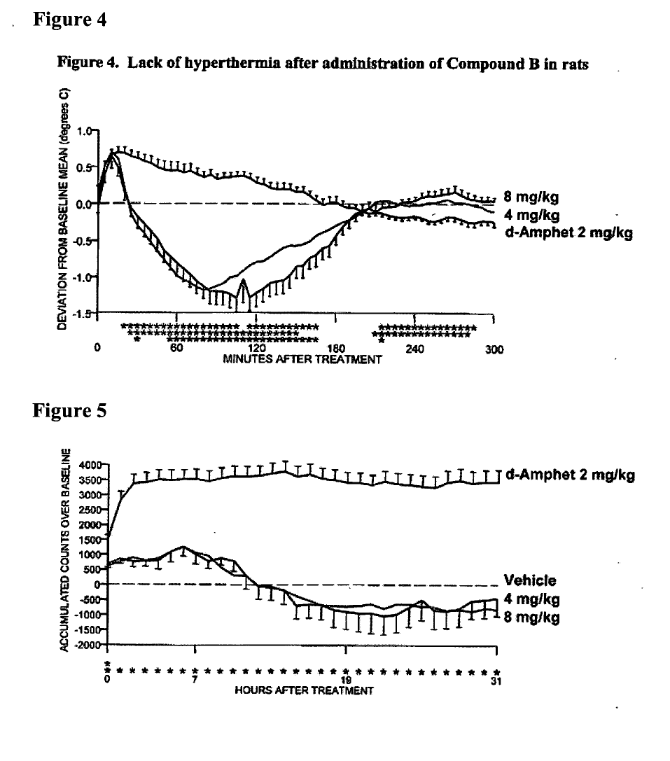

The carbamoyl ester, Compound B, also differed in other unexpected ways from the reference compounds with respect to its effects on body temperature and locomotor activity. Unlike d-amphetamine, Compound B did not cause an increase in body temperature (hyperthermia), but rather caused an opposite reduction in body temperature (hypothermia; FIG. 4).

Moreover, unlike d-methamphetamine, Compound B did not cause locomotor hyperactivity, indicating an absence of stimulant activity (FIG. 5).

Further evidence for a lack of psychostimulant activity in Compound B is given in Example 19.

### Example 19

### Lack of Psychostimulant-Like Effects of Carbamoyl Esters in Drug Discrimination Paradigm

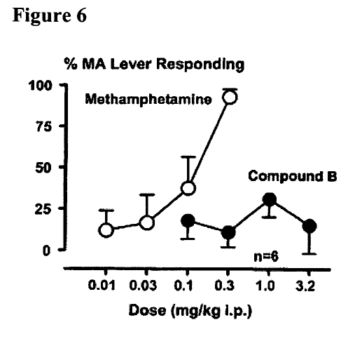

Drug discrimination is an operant paradigm that enables assessment of drug abuse liability (Yasar & Bergman, 1994. Amphetamine-like effect of 1-deprenyl (selegiline) in drug discrimination studies. Clin. Pharmacol. Therap. 56 (S78), 768-773). In this paradigm, psychostimulant properties of novel compounds may be determined in rats trained to discriminate methamphetamine from saline. Hungry rats are initially placed in a test apparatus where they learn that pressing either of two levers results in delivery of a food pellet. Once lever pressing has been established, rats learn that if they are pretreated with methamphetamine, they must now choose (for example) the left hand lever in order to obtain food. On other days, rats are pretreated with vehicle, and must then select the opposite lever in order to obtain food. In this way, rats must learn to use the interoceptive cues generated by the psychostimulant drug to guide its choice of levers. Once lever pressing to a predetermined criterion has been established, a test compound can be administered. On these days, pressing either lever results in food, permitting examination of whether the rats select the methamphetamine or the saline lever. If the rat chooses the methamphetamine lever, the test drug is said to have shown stimulus generalization; that is, it is perceived to be methamphetamine-like by the rat. After administration of Compound B (0.1-3.2 mg/kg i.p., FIG. 6; or 0.32-10 mg/kg p.o.), rats did not select the methamphetamine lever, indicating a lack of stimulus generalization. These findings suggest that Compound B may possess wake promoting activity without psychostimulant drug abuse liability.

### EQUIVALENTS

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of promoting wakefulness in an individual comprising administering to the individual a compound having the formula: 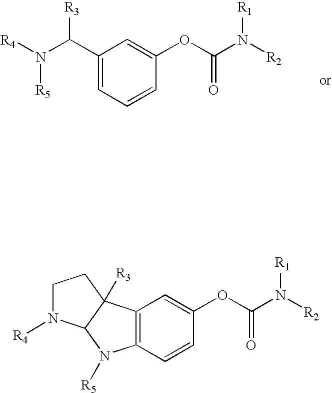
or a pharmaceutically acceptable salt thereof, wherein
R₁ is selected from the group consisting of hydrogen, unsubstituted alkyl, and substituted alkyl;
R₂ is selected from the group consisting of substituted alkyl, unsubstituted aralkyl, substituted aralkyl, unsubstituted heteroaralkyl, substituted heteroaralkyl, unsubstituted heteroaralkyl, substituted heteroaralkyl, unsubstituted aryl, substituted aryl, unsubstituted heteroaryl, substituted heteroaryl, unsubstituted cycloalkyl, substituted cycloalkyl, unsubstituted heterocycloalkyl and substituted heterocycloalkyl;
or taken together with the nitrogen atom to which they are attached, R₁ and R₂ form a 5- or 6-membered ring, further wherein the ring is substituted or unsubstituted;
R₃ is selected from the group consisting of hydrogen, unsubstituted alkyl, and substituted alkyl;
R₄ is selected from the group consisting of hydrogen, unsubstituted alkyl, and substituted alkyl; and
R₅ is selected from the group consisting of hydrogen, unsubstituted alkyl, and substituted alkyl.

2. The method of claim 1, comprising a compound or salt thereof, wherein at least one of R₃, R₄, and R₅ is unsubstituted alkyl.

3. The method of claim 1, comprising a compound or salt thereof, wherein R₁ is hydrogen.

4. The method of claim 1, comprising a compound or salt thereof, wherein R₁ and R₂ taken together with the nitrogen atom to which they are attached form a 5- or 6-membered ring.

5. The method of claim 4, comprising a compound or salt thereof, wherein the 6-membered ring is selected from the group consisting of piperidine and piperazine.

6. The method of claim 1, comprising a compound or salt thereof, wherein R₂ is selected from the group consisting of aralkyl, cycloalkyl, alkyl, and heteroaralkyl, further wherein R₂ is optionally substituted.

7. The method of claim 6, comprising a compound or salt thereof, wherein R₂ is substituted with substituted alkyl, unsubstituted alkyl, substituted cycloalkyl, unsubstituted cycloalkyl, substituted aryl, unsubstituted aryl, substituted tricyclic ring, unsubstituted tricyclic ring, substituted alkenyl-tricyclic ring, unsubstituted alkenyl-tricyclic ring, unsubstituted aryloxy, substituted aryloxy, unsubstituted oxime, and substituted oxime.

8. The method of claim 6, comprising a compound or salt thereof, wherein R₂ is substituted aralkyl.

9. The method of claim 6, comprising a compound or salt thereof, wherein R₂ is substituted alkyl.

10. The method of claim 6, comprising a compound or salt thereof, wherein R₂ is substituted cycloalkyl.

11. The method according to claim 6, comprising a compound or salt thereof, wherein R₂ is substituted heteroaralkyl.

12. A method of promoting wakefulness in an individual comprising administering to the individual a compound of Table 1 or a salt thereof.

13. The method of claim 1, wherein the compound or salt is administered as a pharmaceutical composition comprising a pharmaceutically acceptable carrier.

14. The method of claim 1, wherein the individual suffers from a disorder or condition selected from wakefulness disorders, hypersomnia, sleep apnea, sleep disorders of central origin, fatigue, excessive daytime sleepiness associated with narcolepsy, fatigue and excessive sleepiness associated with a depressive disorder or with antidepressant therapy.

15. The method of claim 14, wherein the wakefulness disorder or condition is selected from circadian rhythm disorder and fatigue associated with multiple sclerosis.

16. The method of claim 15, wherein the circadian rhythm disorder is selected from shift work sleep disorder, sleep apnea, desynchronizing disorder in blind individuals, time zone change syndrome, shift work sleep disorder, irregular sleep pattern, delayed sleep syndrome, and advanced sleep syndrome.

17. A method for enhancing alertness or increasing regularity of sleep rhythms in an individual comprising administering to the individual a compound having the formula: 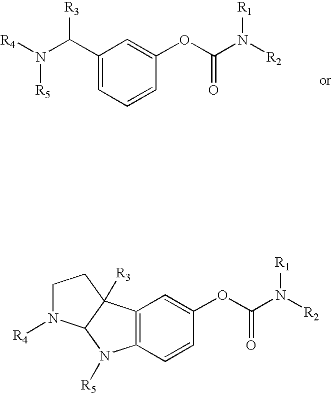
or a pharmaceutically acceptable salt thereof,
R₁ is selected from the group consisting of hydrogen, unsubstituted alkyl, and substituted alkyl;
R₂ is selected from the group consisting of substituted alkyl, unsubstituted aralkyl, substituted aralkyl, unsubstituted heteroaralkyl, substituted heteroaralkyl, unsubstituted heteroaralkyl, substituted heteroaralkyl, unsubstituted aryl, substituted aryl, unsubstituted heteroaryl, substituted heteroaryl, unsubstituted cycloalkyl, substituted cycloalkyl, unsubstituted heterocycloalkyl and substituted heterocycloalkyl;
or taken together with the nitrogen atom to which they are attached, R₁ and R₂ form a 5- or 6-membered ring, further wherein the ring is substituted or unsubstituted;
R₃ is selected from the group consisting of hydrogen, unsubstituted alkyl, and substituted alkyl;
R₄ is selected from the group consisting of hydrogen, unsubstituted alkyl, and substituted alkyl; and
R₅ is selected from the group consisting of hydrogen, unsubstituted alkyl, and substituted alkyl.

18. The method of claim 1, wherein the compound or salt thereof is administered enterally, parenterally, orally or intramuscularly.

19. The method of claim 1, wherein the minimum effective dose (MED) of the compound or pharmaceutically acceptable salt is ≦8 mg/kg p.o.

20. A kit for carrying out a method of promoting wakefulness in an individual comprising administering to the individual a compound having the formula: 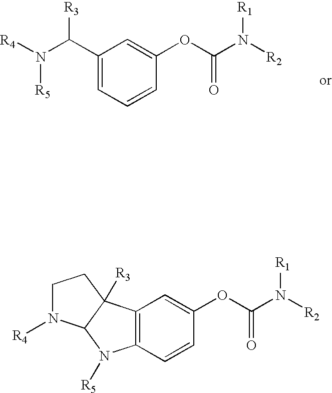
or a pharmaceutically acceptable salt thereof, wherein
R₁ is selected from the group consisting of hydrogen, unsubstituted alkyl, and substituted alkyl;
R₂ is selected from the group consisting of substituted alkyl, unsubstituted aralkyl, substituted aralkyl, unsubstituted heteroaralkyl, substituted heteroaralkyl, unsubstituted heteroaralkyl, substituted heteroaralkyl, unsubstituted aryl, substituted aryl, unsubstituted heteroaryl, substituted heteroaryl, unsubstituted cycloalkyl, substituted cycloalkyl, unsubstituted heterocycloalkyl and substituted heterocycloalkyl;
or taken together with the nitrogen atom to which they are attached, R₁ and R₂ form a 5- or 6-membered ring, further wherein the ring is substituted or unsubstituted;
R₃ is selected from the group consisting of hydrogen, unsubstituted alkyl, and substituted alkyl;
R₄ is selected from the group consisting of hydrogen, unsubstituted alkyl, and substituted alkyl; and
R₅ is selected from the group consisting of hydrogen, unsubstituted alkyl, and substituted alkyl.
